(19) United States
(12) Patent Application Publication
(10) Pub. No.: US 2026/0125342 A1
(43) Pub. Date: May 7, 2026

(54) SYNTHESIS OF ACID HYDRAZIDES USING FLOW CHEMISTRY

(71) Applicant: The Regents of the University of California, Oakland, CA (US)

(72) Inventors: Matthew W. Halloran, San Diego, CA (US); Michael D. Burkart, San Diego, CA (US); Caitlin Hudecek, San Diego, CA (US)

(21) Appl. No.: 19/437,555

(22) Filed: Dec. 31, 2025

Related U.S. Application Data
(63) Continuation of application No. PCT/US2024/036541, filed on Jul. 2, 2024.
(60) Provisional application No. 63/512,050, filed on Jul. 5, 2023.

(51) Int. Cl. C07C 241/04

(52) U.S. Cl. CPC C07C 241/04

(57) ABSTRACT

A continuous process for preparing a compound of Formula (III)

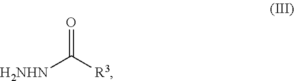

or a salt and/or solvate thereof, includes forming a first reaction stream comprising a compound of Formula (I) dissolved in an alcohol;

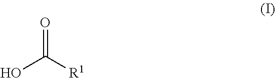

flowing the reaction stream from step (a) through a continuous flow reactor at a temperature of about 100° C. to about 160° C. and at a flow rate of about 0.2 mL/min to about 5 mL/min; contacting the reaction stream from step (b) with a second reaction stream comprising a hydrazine source to form a combined reaction stream; and (d) flowing the combined reaction stream from step (c) through a continuous flow reactor at a temperature of about 100° C. to about 160° C. and at a flow rate of about 0.2 mL/min to about 5 mL/min.

### CROSS-REFERENCE TO RELATED APPLICATIONS

This application is a U.S. Continuation of PCT International Patent Application No. PCT/US2024/036541, filed Jul. 2, 2024, which claims the benefit of priority under 35 U.S.C. § 119(e) to U.S. Provisional Ser. No. 63/512,050 filed Jul. 5, 2023, the contents of each of which are hereby incorporated by reference into the present disclosure in their entirety for any and all purposes.

### STATEMENT OF GOVERNMENT SUPPORT

This invention was made with government support under DE-EE0008246 awarded by the U.S. Department of Energy. The government has certain rights in the invention.

### BACKGROUND

Acid hydrazides are a versatile class of organic compounds which serve as fundamental building blocks in a number of pharmaceutical, agrochemical, and material science applications. In addition, they function as valuable chemical intermediates in the synthesis of ketones, hydrazones, amides, and heterocyclic rings. As a subset of this family, acid dihydrazides find significant applications in a variety of industrial and academic settings owing to their benchtop stability and unique reactivity profile. Accordingly, they are commonly used as curing agents for epoxy resins, corrosion inhibitors, biomimetic hydrogels, or as precursors for the synthesis of acyl azides and diisocyanates.

### SUMMARY OF THE DISCLOSURE

The present disclosure provides methods of preparing hydrazides from carboxylic acids in continuous flow.

In a first aspect, the present disclosure provides a continuous process for preparing a compound of Formula (III)

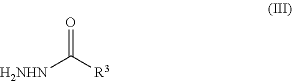

or a salt and/or a solvate thereof, comprising: (a) forming a first reaction stream comprising a compound of Formula (I)

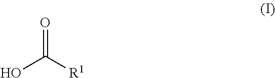

dissolved in an alcohol; (b) flowing the reaction stream from step (a) through a continuous flow reactor at a temperature of about 100° C. to about 160° C. and at a flow rate of about 0.2 mL/min to about 5 mL/min; (c) contacting the reaction stream from step (b) with a second reaction stream comprising a hydrazine source to form a combined reaction stream; and (d) flowing the combined reaction stream from step (c) through a continuous flow reactor at a temperature of about 100° C. to about 160° C. and at a flow rate of about 0.2 mL/min to about 5 mL/min; wherein R¹ is Y or

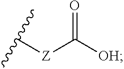

### R³ is Y or

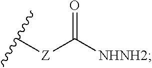

Y is an aryl, heteroaryl, cycloalkyl, or heterocycloalkyl group; Z is a divalent group selected from arylene, heteroarylene, alkylene, cycloalkylene, and heterocycloalkylene groups; and R¹ and R³ are both Y, or R¹ and R³ are both not Y. In some embodiments of this first aspect, the compound of Formula (I) is converted to a compound of Formula (II)

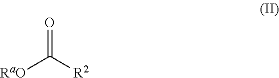

in step (b), wherein: R² is Y or

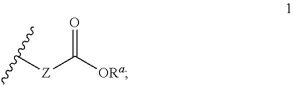

and when R¹ and R³ are both Y, R² is Y; and when R¹ and R³ are both not Y, R² is not Y. In some embodiments of this first aspect, the flow rate in step (b) provides a residence time of about 2 minutes to about 15 minutes. In some embodiments of this first aspect, the flow rate in step (b) provides a residence time of about 5.3 minutes or about 9 minutes. In some embodiments of this first aspect, the continuous process further comprises cooling the reaction stream from step (b) to about 0° C. before step (c). In further embodiments of this first aspect, the cooling step comprises flowing the reaction stream from step (b) through an ice bath with a residence time of less than about 1 minute, or less than 0.5 minutes. In some further embodiments of this first aspect, the residence time in the cooling step is about 0.25 minutes. In some embodiments of this first aspect, the hydrazine source in step (c) is hydrazine hydrate. In some embodiments of this first aspect, the hydrazine source is present in the second reaction stream in an amount of about 2 equivalents to about 5 equivalents relative to the amount of —COOH functional group in the compound of Formula (I). In some embodiments of this first aspect, the flow rate is step (d) provides a residence time of about 2 minutes to about 15 minutes. In some embodiments of this first aspect, the flow rate in step (d) provides a residence time of about 6.6 minutes or about 13.2 minutes. In some embodiments of this first aspect, the continuous process further comprises (e) flowing the reaction stream from step (d) through a backpressure regulator; and (f) cooling the reaction stream from step (e) to about 60° C. In some further embodiments of this first aspect, the cooling step comprises flowing the reaction stream from step (e) through a continuous flow reactor at a temperature of about 60° C., with a residence time of less than about 1 minute. In some embodiments of this first aspect, the continuous process further comprises collecting a reactor effluent exiting from the continuous flow reactor. In some further embodiments of this first aspect, the continuous process further comprising cooling the reactor effluent existing from the continuous flow reactor, thereby forming precipitates of a compound of Formula (III). In some embodiments of this first aspect, the continuous process further comprises isolating the compound of Formula (III). In some further embodiments of this first aspect, the continuous process further comprises collecting any remaining material after the compound of Formula (III) is isolated, and repeating step (a) using said remaining material. In some embodiments of this first aspect, the compound of Formula (I) is a compound having a formula of

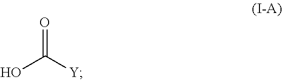

and the compound of Formula (III) is a compound having the formula of

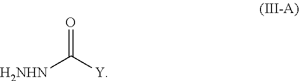

In some further embodiments of this first aspect, the compound of Formula (I-A) is converted to a compound of Formula (II-A)

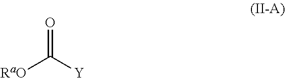

in step (b). In some embodiments of this first aspect, Y is selected from the group consisting of isoxazolyl, phenyl, alkyl, and cycloalkyl. In some embodiments of this first aspect, Y is selected from the group consisting of

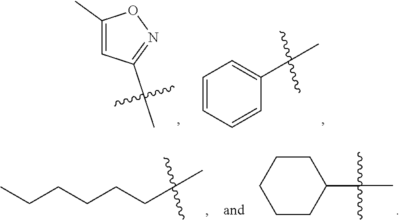

In some embodiments of this first aspect the compound of Formula (I) is a compound having a formula of

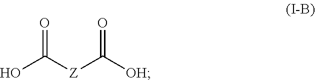

and the compound of Formula (III) is a compound having a formula of

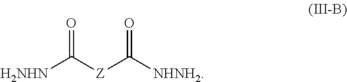

In some further embodiments of this first aspect, the compound of Formula (I-B) is converted to a compound of Formula (II-B)

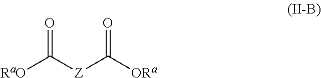

in step (b). In some embodiments of this first aspect, Z is selected from the group consisting of phenylene, alkylene, and cycloalkylene. In some embodiments of this first aspect, Z is selected from the group consisting of

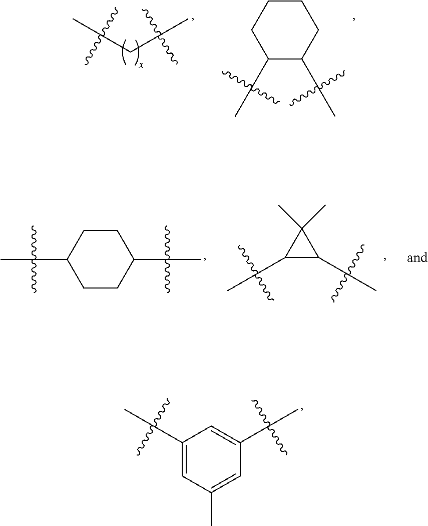

wherein x is an integer from 1-7.

### BRIEF DESCRIPTION OF THE DRAWINGS

FIG. 1 depicts an overview of the findings of the methodology for the synthesis of acid hydrazides from carboxylic acids via a continuous flow process in yields ranging from 65-91% with a residence time of between 13 to 25 minutes, according to various embodiments.

FIGS. 2A and 2B show the applicability of acid hydrazide functionality and a synthesis scheme, according to various embodiments. FIG. 2A depicts the structure of various acid hydrazide examples along with their applications. FIG. 2B depicts the synthesis of renewable isocyanates in continuous flow via in situ acyl azide generation from corresponding hydrazides followed by a thermally induced Curtis rearrangement.

FIG. 3 depicts the standard Fischer esterification conditions that could be adapted to continuous flow, according to various embodiments.

FIG. 4 depicts the optimized continuous flow hydrazinolysis method of dimethyl azelate.

FIG. 5 depicts the telescoped synthesis of acid hydrazides from carboxylic acids in flow.

FIG. 6 depicts the esterification of azelaic acid flow diagram.

FIG. 7 depicts the hydrazinolysis of dimethyl azelate flow diagram.

FIG. 8 depicts the optimized hydrazinolysis of dimethyl azelate flow diagram.

FIG. 9 depicts a flow scheme of continuous synthesis of acid hydrazides from carboxylic acids corresponding to General Procedure A.

FIG. 10 depicts a flow scheme of continuous synthesis of acid hydrazides from carboxylic acids corresponding to General Procedure B.

### DETAILED DESCRIPTION

### Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

All temperatures are in degrees Celsius (° C.) unless otherwise specified.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free (e.g., contains less than 5 wt %, 4 wt %, 3 wt %, 2 wt %, or 1 wt %) of other components. As used herein, calculation of percent purity of isolated substances and/or entities should not include excipients (e.g., buffer, solvent, water, etc.).

Approximately or about: as used herein, the term “approximately” or “about”, as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used in the description and the appended claims, the singular forms “a”, “an”, and “the” include plural referents unless the context clearly dictates otherwise. Thus, for example, reference to “a composition” includes mixtures of two or more such compositions.

Throughout the description and claims of this specification the word “comprise” and other forms of the word, such as “comprising” and “comprises”, means including but not limited to, and is not intended to exclude, for example, other additives, components, integers, or steps.

“Optional” or “optionally” means that the subsequently described event or circumstance may or may not occur, and that the description includes instances where the event or circumstance occurs and instances where it does not.

Tautomer: refers to a molecule wherein a proton shift from one atom of a molecule to another atom of the same molecule is possible. The compounds presented herein may, in certain embodiments, exist as tautomers. In circumstances where tautomerization is possible, a chemical equilibrium of the tautomers will exist. The exact ratio of the tautomers depends on several factors, including physical state, temperature, solvent, and pH.

Solvate: can include, but is not limited to, a solvate that retains one or more of the activities and/or properties of the compound and that is not undesirable. Examples of solvates include, but are not limited to, a compound in combination with water, isopropanol, ethanol, methanol, DMSO, ethyl acetate, acetic acid, ethanolamine, or combinations thereof.

Salt: as used herein, a salt refers to preferably a salt of a mineral acid, or an organic acid such as a carboxylic acid or a sulfonic acid, and/or to alkali, alkaline earth, and various ammonium (including tetraalkyl ammonium, pyridinium, imidazolium and the like) salts.

Solvent: can include, but is not limited to, non-polar, polar aprotic, and polar protic solvents. Illustrative examples of non-polar solvents include, but are not limited to, pentane, cyclopentane, hexane, cyclohexane, benzene, toluene, xylene, 1,4-dioxane, chloroform, diethyl ether, and dichloromethane (DCM). Illustrative examples of polar aprotic solvents include, but are not limited to, tetrahydrofuran (THF), ethyl acetate, isopropyl acetate (IPAc), acetone, dimethylformamide (DMF), dimethyl acetamide (DMAc), acetonitrile (MeCN), butyronitrile, dimethyl sulfoxide (DMSO), nitromethane, and propylene carbonate. Illustrative examples of polar protic solvents include, but are not limited to, formic acid, n-butanol, isopropanol (IPA), n-propanol, ethanol, methanol, acetic acid, and water.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SFs), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; and nitriles (i.e., CN).

Substituted ring groups such as substituted cycloalkyl, aryl, acyl, heterocyclyl and heteroaryl groups also include rings and ring systems in which a bond to a hydrogen atom is replaced with a bond to a carbon atom. Therefore, substituted cycloalkyl, aryl, acyl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Cycloalkyl groups may be substituted or unsubstituted. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. Heterocyclyl groups may be substituted or unsubstituted. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. The phrase includes heterocyclyl groups that have other groups, such as alkyl, oxo or halo groups, bonded to one of the ring members, referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups may be substituted or unsubstituted. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Representative substituted heteroaryl groups may be substituted one or more times with various substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation.

The terms “alkanoyl” and “alkanoyloxy” as used herein can refer, respectively, to —C(O)-alkyl groups and —O—C(O)-alkyl groups, each containing 2-5 carbon atoms. Similarly, “aryloyl” and “aryloyloxy” refer to —C(O)-aryl groups and —O—C(O)-aryl groups.

The term “carboxylate” as used herein refers to a —COOH group or its ionized salt form.

The term “ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O⁻. A “hydroxyalkyl” group is a hydroxyl-substituted alkyl group, such as HO—CH₂—.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Those of skill in the art will appreciate that compounds of the present technology may exhibit the phenomena of tautomerism, conformational isomerism, geometric isomerism, and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

Continuous flow chemistry is an emerging technology with desirable features compared to conventional batch manufacturing processes. Continuous flow chemistry allows for faster and safer reaction, which can result in products of better quality and can permit the performance of chemistry that is difficult to conduct through batch processing methods. These methods are characterized by streams of reactants that are introduced at specific flow rates to a single chamber where the chemical reaction occurs and then the resulting compounds are collected and directed to subsequent reactor loops to generate a final product. The streams generally introduce small amounts of reagents at one time, which increases safety, but the method allows for a consistent flow of the reagents, which increases production volume overall. Described herein are continuous methods of preparing acid hydrazides or a salt and/or solvate thereof.

Disclosed herein are methods and continuous processes of preparing acid hydrazides (e.g., a compound of Formula (III)

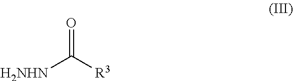

or a salt and/or solvate thereof) from carboxylic acids (e.g., a compound of Formula (I)

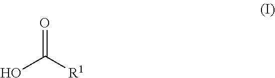

or a salt and/or solvate thereof) in continuous flow. The processes described herein can produce the acid hydrazide products in yields ranging from 65-91%. In particular, the processes described herein can be performed using short residence times of between 13-25 minutes, and the processes can covert a variety of mono- and diacids, including aliphatic, aromatic, and heteroaromatic functionalities, to the corresponding acid hydrazide products. Further, large-scale synthesis (e.g., 200 g) can be achieved using the described processes.

The method and continuous process may be used to prepare an acid hydrazide product from a carboxylic acid. The acid hydrazide may have the structure of Formula (III)

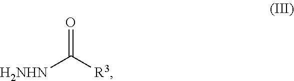

wherein R³ may be Y or

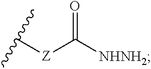

Y may be an aryl, heteroaryl, alkyl, cycloalkyl, or heterocycloalkyl group; and Z may be a divalent group selected from arylene, heteroarylene, alkylene, cycloalkylene, and heterocycloalkylene groups. Further, Y may be selected from the group consisting of isoxazolyl, phenyl, alkyl, and cycloalkyl. Y may be selected from the group consisting of

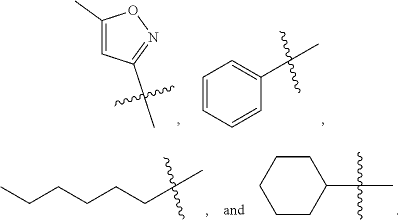

Further, Z may be selected from the group consisting of phenylene, alkylene, and cycloalkylene. Z may be selected from the group consisting of

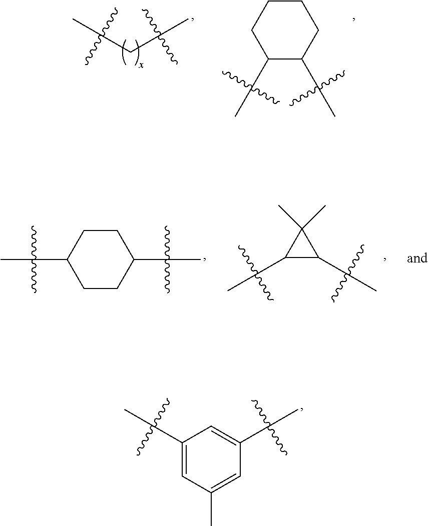

wherein x is an integer from 1-7. More specifically, the acid hydrazide may have the structure of Formula (III-A)

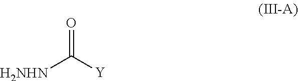

### or Formula (III-B)

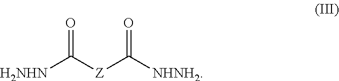

The acid hydrazide product may be a salt and/or a solvate of the acid hydrazide.

The carboxylic acid may have the structure of Formula (I)

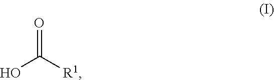

wherein R¹ may be Y or

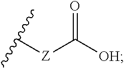

Y may be an aryl, heteroaryl, alkyl, cycloalkyl, or heterocycloalkyl group; and Z may be a divalent group selected from arylene, heteroarylene, alkylene, cycloalkylene, and heterocycloalkylene groups. Further, Y may be selected from the group consisting of isoxazolyl, phenyl, alkyl, and cycloalkyl. Y may be selected from the group consisting of

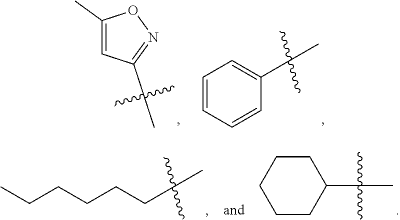

Further, Z may be selected from the group consisting of phenylene, alkylene, and cycloalkylene. Z may be selected from the group consisting of

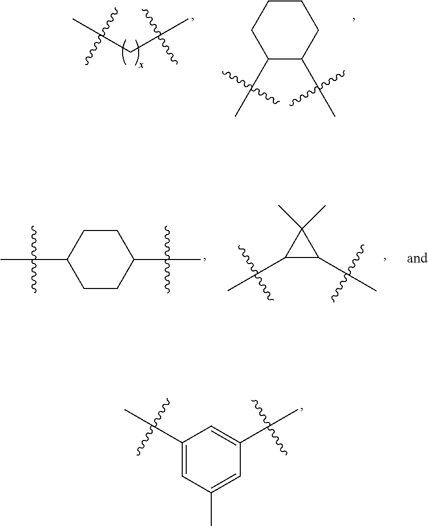

wherein x is an integer from 1-7. More specifically, the carboxylic acid may have the structure of

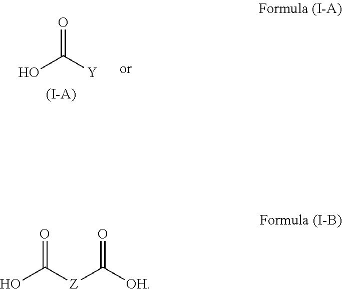

In an embodiment, R¹ and R³ are both Y. Alternatively, in an embodiment, R¹ and R² and both not Y.

The method and continuous process may include the step of forming a first reaction stream comprising the carboxylic acid described above dissolved in alcohol. The alcohol of the first reaction stream may have a formula of R^{a}OH, wherein R^{a} is a C₁-C₆ alkyl. More specifically, R^{a} may be methyl. The first reaction stream may further include an acid. The acid may be present in an amount of less than 0.5 equivalent (e.g., about 0.1 to about 0.3 equivalent, such as about 0.2 equivalent) relative to the amount of a compound of Formula (I). The acid in the first reaction stream may be a strong acid. More specifically, the acid may be H₂SO₄. The compound of Formula (I) may have a concentration of about 0.2 M to about 2 M in the first reaction stream. This may include, but is not limited to, ranges such as from about 1 M to about 2M, or at about 1.5 M.

The method and continuous process may next include the step of flowing the first reaction stream through a continuous flow reactor at a temperature of about 100° C. to about 160° C. and at a flow rate of about 0.2 mL/min to about 5 mL/min. The temperature may be about 130° C. to about 140° C. This may include, in some embodiments, a reaction temperature of 135° C. The flow rate may be about 1.5 mL/min to about 2.5 mL/min (e.g., about 2 mL/min) or about 0.5 mL/min to about 1.5 mL/min (e.g., about 1 mL/min). In some embodiments, the flow rate may provide a residence time of about 2 minutes to about 15 minutes. More specifically, the flow rate may provide a residence time of about 5 minutes to about 6 minutes (e.g., about 5.3 minutes) or about 8.5 minutes to about 9.5 minutes (e.g., about 9 minutes). In this step, the compound of Formula (I) may be converted to a compound of Formula (II),

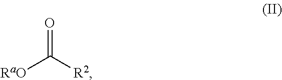

wherein R² is Y or

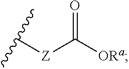

and when R¹ and R³ are both Y, R² is Y; and when R¹ and R3 are both not Y, R² is not Y. In some embodiments, when the compound of Formula (I) has the structure of Formula (I-A), the compound of Formula (I-A) may be converted to a compound of Formula (II-A)

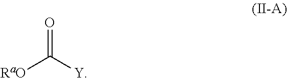

In some embodiments, when the compound of Formula (I) has the structure of Formula (I-B), the compound of Formula (I-A) may be converted to a compound of Formula (II-B)

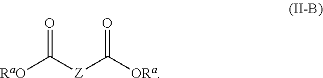

The method and continuous process may next include the step of contacting the reaction stream from the previous step with a second reaction stream comprising a hydrazine source to form a combined reaction stream. In some embodiments, the reaction stream may be subjected to a cooling step prior to the contacting step. The cooling step may be cooling the reaction stream from the previous step to about 0° C. before the contacting step. The cooling step may include flowing the reaction stream from the previous step through an ice bath with a residence time of less than about 1 minute or less than 0.5 minutes. In some embodiments, the residence time in the cooling step may be about 0.25 minutes. The second reaction stream may include an alcohol solvent. More specifically, the alcohol solvent may be methanol. The hydrazine source in the second reaction stream may be hydrazine hydrate. The hydrazine source may be present in the second reaction stream in an amount of about 2 equivalents to about 5 equivalents (e.g., about 3 equivalents to about 3.5 equivalents, such as about 3.2 equivalents) relative to the amount of —COOH functional group in the compound of Formula (I). The hydrazine source may have a concentration of about 2 M to about 10 M.

The method and continuous process may next include the step of flowing the combined reaction stream from the previous step through a continuous flow reactor at a temperature of about 100° C. to about 160° C. and at a flow rate of about 0.2 mL/min to about 5 mL/min. In some embodiments, the temperature may be about 120° C. to about 130° C. (e.g., about 125° C.) or about 100° C. to about 140° C. (e.g., about 135° C.). The flow rate may be about 1.5 mL/min to about 2.5 mL/min (e.g., 2 mL/min) or about 0.5 mL/min to about 1.5 mL/min (e.g., about 1 mL/min). In some embodiments, the flow rate may provide a residence time of about 2 minutes to about 15 minutes. The flow rate may provide a resistance time of about 6 minutes to about 7 minutes (e.g., 6.6 minutes) or about 13 minutes to about 14 minutes (e.g., about 13.2 minutes). The method and continuous process may optionally include the additional steps of flowing the reaction stream from the previous step through a backpressure regulator. The method and continuous process may also include cooling the reaction stream from the backpressure regulator to about 60° C. The backpressure regulator may be a 250 psi backpressure regulator. The cooling step may include flowing the reaction stream through a continuous flow reactor at a temperature of about 60° C. and with a residence time of less than about 1 minute. The residence time in the cooling step may be about 0.5 minutes.

The method and continuous process may include the step of collecting a reactor effluent exiting the continuous flow reactor, and optionally, cooling the reactor effluent exiting from the continuous flow reactor, thereby forming precipitates of a compound of Formula (III). The method and continuous process may include isolating the compound of Formula (III) and, optionally, collecting any remaining material after the compound of Formula (III) is isolated and repeating the first forming step and, optionally, the whole method, using said remaining material.

A range of concentrations of reactants (and/or reagents) may be used in the present methods. Typically, concentrations would be selected to achieve the highest yield of the compound of interest. For example, the concentration of a reactant (and/or reagent) described herein may be about 0.05 molar (M) to about 20 M, such as about 0.05 M, about 0.1 M, about 0.2 M, about 0.3M, about 0.4M, about 0.5 M, about 0.6 M, about 0.7 M, about 0.8 M, about 0.9 M, about 1 M, about 1.1 M, about 1.2 M, about 1.3 M, about 1.4 M, about 1.5 M, about 2 M, about 3 M, about 4 M, about 5 M, about 6 M, about 7 M, about 8 M, about 9 M, about 10 M, about 12 M, about 14 M, about 16 M, about 18 M, about 20 M, or in a range between and including any two of the foregoing values.

In any embodiments of the methods herein, the reaction may be performed at a temperature from about 80° C. to about 160° C., such as about 80° C., about 100° C., about 110° C., about 120° C., about 125° C., about 130° C., about 135° C., about 140° C., about 145° C., about 150° C., about 155° C., about 160° C., or a range between or including any two of the foregoing values.

In any embodiments herein, a range of flow rate may be employed for a continuous process described herein, depending on reaction scale and conditions. For example, the flow rate may range from about 0.2 mL/min to about 10 mL/min, such as about 0.2 mL/min, about 0.5 mL/min, about 1 mL/min, about 1.5 mL/min, about 2 mL/min, about 2.5 mL/min, about 3 mL/min, about 3.5 mL/min, about 4 mL/min, about 4.5 mL/min, about 5 mL/min, about 6 mL/min, about 7 mL/min, about 8 mL/min, about 9 mL/min, about 10 mL/min, or a range between or including any two of the foregoing values.

In any embodiments herein, a flow rate described herein may provide a suitable residence time (e.g., a residence time sufficient to convert a reactant to the corresponding product, or a residence time sufficient to reduce (or increase) the temperature of a reaction mixture). Accordingly, in any embodiment herein, the residence time may be about 0.1 minutes to about 20 minutes, such as about 0.1 minutes, about 0.25 minutes, about 0.5 minutes, about 0.75 minutes, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 18 minutes, about 20 minutes, or a range between or including any two of the foregoing values.

In any embodiments, the present processes may further comprise isolating and/or purifying the product formed (e.g., a compound of Formula (III)) from the reaction mixture. In any embodiments, the present methods may comprise subjecting the product of a compound of Formula (III) to further reactions.

In any embodiments, the present processes may provide a compound of Formula (III) from a compound of Formula (I) in more than 50% yield (e.g., 65-91% yield), including more than 60% yield, more than 80% yield. In any embodiments, the present processes may be performed using short residence times (e.g., less than 40 minutes, or less than 30 minutes, such as between 13-25 minutes).

Thus, in one aspect, provided herein is a continuous process for preparing a compound of Formula (III)

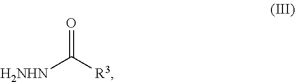

or a salt and/or solvate thereof, comprising:
(a) forming a first reaction stream comprising a compound of Formula (I) dissolved in an alcohol;

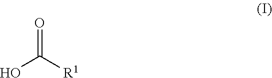

(b) flowing the reaction stream from step (a) through a continuous flow reactor at a temperature of about 100° C. to about 160° C. and at a flow rate of about 0.2 mL/min to about 5 mL/min;
(c) contacting the reaction stream from step (b) with a second reaction stream comprising a hydrazine source to form a combined reaction stream;
(d) flowing the combined reaction stream from step (c) through a continuous flow reactor at a temperature of about 100° C. to about 160° C. and at a flow rate of about 0.2 mL/min to about 5 mL/min; wherein
   R¹ is Y

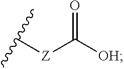

R³ is Y or

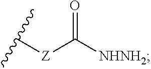

Y is an aryl, heteroaryl, alkyl, cycloalkyl, or heterocycloalkyl group;
Z is a divalent group selected from arylene, heteroarylene, alkylene, cycloalkylene, and heterocycloalkylene groups; and
R¹ and R³ are both Y, or R¹ and R³ are both not Y.

### Examples

It will be apparent to those skilled in the relevant art that various modifications, additions, substitutions, and the like can be performed without altering the spirit or scope of the invention, and such modifications and variations are encompassed with invention as defined in the claims which follow. The invention disclosed herein is further illustrated by the examples which in no way should be construed as being limiting.

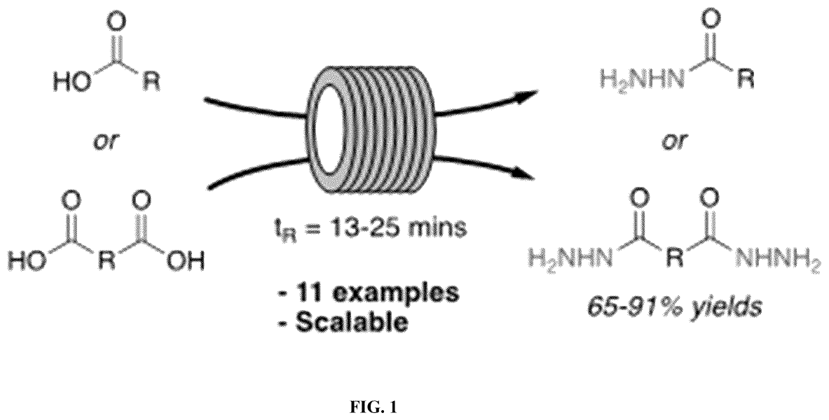

Example 1. Synthesis of Acid Hydrazides from Carboxylic Acids in Continuous Flow. A methodology for the synthesis of acid hydrazides from carboxylic acids via a continuous flow process in yields ranging from 65-91% is described generally in FIG. 1. Using short residence times of between 13-25 minutes, the conditions are applicable to a variety of mono- and diacids, including aliphatic, aromatic, and heteroaromatic functionalities. To demonstrate scalability, a large scale (200 g) synthesis of azelaic dihydrazide was conducted over a continuous run of 9 hours with an 86% overall yield.

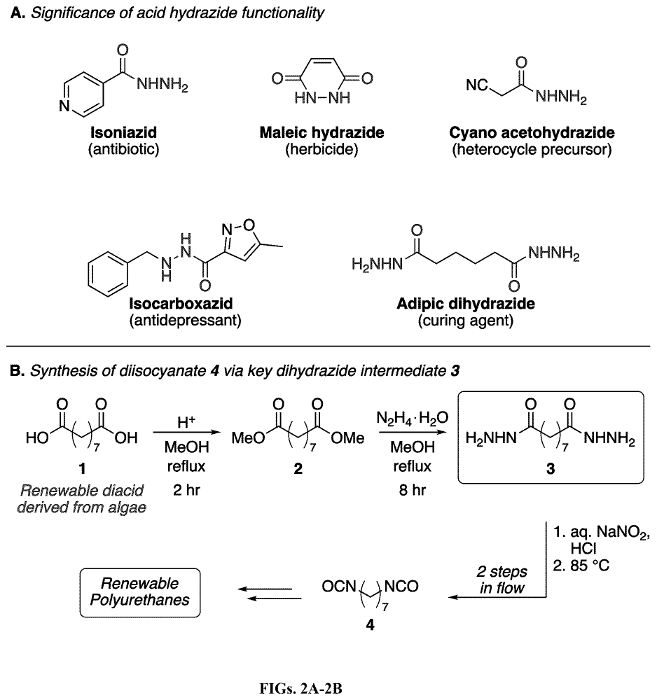

Acid hydrazides are a versatile class of organic compounds which serve as fundamental building blocks in a number of pharmaceutical, agrochemical, and material science applications (FIG. 2A). In addition, they function as valuable chemical intermediates in the synthesis of ketones, hydrazones, amides, and heterocyclic rings. As a subset of this family, acid dihydrazides find significant applications in a variety of industrial and academic settings owing to their benchtop stability and unique reactivity profile. Accordingly, they are commonly used as curing agents for epoxy resins, corrosion inhibitors, biomimetic hydrogels, or as precursors for the synthesis of acyl azides and diisocyanates. In this context, a scalable methodology for the synthesis of renewable isocyanates in continuous flow via in situ acyl azide generation from corresponding hydrazides, followed by a thermally induced Curtius rearrangement (FIG. 2B), was recently reported. Using this methodology, a variety of mono- and diisocyanates can be synthesized, the latter used to manufacture polyurethanes with a wide range of material properties.

The present technology addresses the practical need for a scalable process to generate dihydrazide 3 from azelaic acid (1)—a renewably sourced diacid which can be derived from algae-sourced palmitoleic acid via ozonolysis. Traditionally, acid hydrazides are synthesized via a two-step process in batch, wherein a carboxylic (either mono- or di-) acid is first converted to its ester or acid chloride, then treated with hydrazine hydrate at elevated temperatures. Although this process is effective on smaller gram scales in the laboratory, larger scale production of dihydrazides following this approach is both time consuming and energy intensive given the need to work-up and isolate the ester or acid chloride intermediate. Additionally, handling large quantities of hydrazine hydrate at high temperatures in a batch reactor poses safety concerns. While alternative methodologies have been reported, such as one-pot microwave-assisted or carbodiimide-mediated coupling, these strategies suffer from drawbacks, including scalability issues in the former, and poor atom-economy resulting from the use of stoichiometric coupling reagents in the latter. Moreover, both of these methods were limited to the synthesis of monohydrazides in their substrate scope. As an alternative strategy, the use of a continuous flow process would allow for the large-scale production of 3 from 1 in a more controlled and efficient manner that could be directly translatable into an industrial setting.

Over the last fifteen years, flow chemistry has emerged as an important tool for synthetic and process chemists, owing to its superior mass transfer and reaction kinetics, enhanced safety in handling hazardous chemicals, and adaptability to scale up. In line with this, the aim of this work was to develop a methodology for the continuous flow synthesis of acid hydrazides from carboxylic acids, which is suitable for kilogram scale production of dihydrazide.

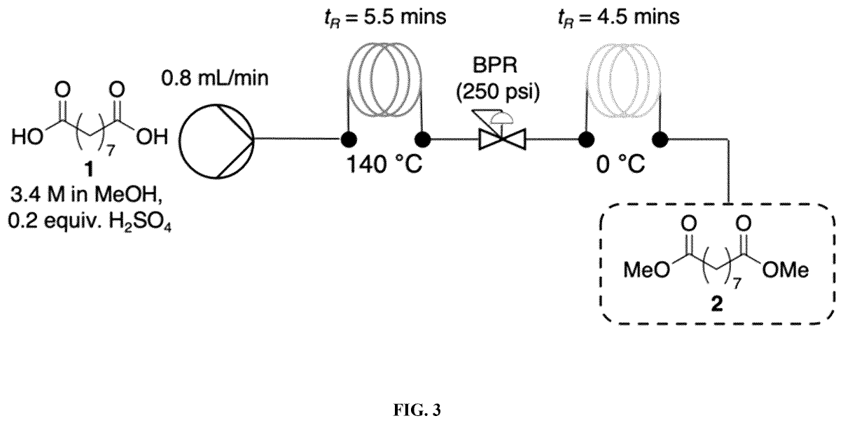

As a starting point, conditions for the Fisher esterification of 1 in flow were screened using commercially available ion-exchange resins Amberlyst 15 and 36 in a packed bed reactor. Disappointingly, neither resin proved acceptable. Even at an elevated temperature of 120° C. and with extended residence times, little to no diester was obtained under relevant flow conditions. Although esterification in continuous flow has been achieved using a variety of techniques, such as other sulfonic acid ion-exchange resins, vortex fluidic devices, and diazotization of amines, most require prolonged residence times, specialized reactors, or are limited to mono-functionalized substrates. In this context, the aim was to identify a set of reaction conditions using standardized flow chemistry equipment that would be amenable to di-functionalized substrates and easily translatable to scale. Gratifyingly, standard Fischer esterification conditions (i.e., catalytic H₂SO₄ in methanol) were found that could be directly adapted to continuous flow. Under these conditions, 1 was rapidly esterified to afford dimethyl azelate 2 in a 93% yield (FIG. 3). The reaction was run on a 5 g scale of 1 to afford 2 in a 93% isolate yield following workup.

Next, attention was turned toward developing suitable conditions for the hydrazinolysis of 2 in continuous flow. In contrast to esterification, the adaptation of hydrazinolysis into flow posed initial problems. During the course of a batch reaction, the dihydrazide product (3) vigorously precipitates out of solution, which can lead to clogging in a tubular reactor or backpressure regulator.

To avoid this, key parameters, such as tubing diameter, flow rate, residence time, and reagent concentration, would need to be carefully selected and optimized. 3 the immediate goal of scaling this process, ideal reaction conditions would furnish 3 as a precipitate in suspension, which could be isolated by filtration and without the need for further purification.

To avoid potential precipitation and clogging issues in the tubular reaction, initial runs were conducted under dilute reagent conditions of <0.4 M. It was found that the reaction proceeded smoothly under continuous flow conditions to provide 3 in an 88% yield with no precipitation occurring in the tubing (Table 1). While promising, the isolation of 3 required the use of a rotary evaporator to concentrate the collected reaction mixture, a process which would be both energy and time intensive at scale. Attempts at increasing the concentration of 2 were met with difficulty, as copious amounts of 3 precipitated within the tubing immediately after the backpressure regulator. It was reasoned that this precipitation was occurring due to the rapid temperature and pressure drops that the solution was experiencing as it exited the backpressure regulator at 125° C. and immediately cooled to 0° C. at atmospheric pressure, causing precipitation of 3 to occur in an uncontrolled manner.

TABLE 1
Result of Reaction Conditions

| | Diester | | | Total | | Observations | |
|---|---|---|---|---|---|---|---|
| | Conc. In | | | Flow | | during | |
| | Reactor | Equiv. | t_{R} | Rate | Tₒᵤₜₗₑₜ | Reaction (in | Yield of 3 |
| Entry | (M) | N₂H₄•H₂O | (min) | (mL/min) | (° C.) | tubing) | (%) |
| 1^{a} | 0.2 | 10 | 12 | 2 | 0 | No | 88 (rotary |
| | | | | | | precipitate | evaporator) |
| 2^{a} | 0.4 | 10 | 12 | 2 | 0 | No | 84 (rotary |
| | | | | | | precipitate | evaporator) |
| 3^{b} | 0.6 | 10 | 12 | 2 | 0 | Precipitate | — |
| | | | | | | forms - | |
| | | | | | | clogged | |
| | | | | | | tubing | |
| 4^{b} | 0.5 | 10 | 12 | 2 | 0 | Precipitate | — |
| | | | | | | forms - | |
| | | | | | | clogged | |
| | | | | | | tubing | |
| 5^{b} | 0.5 | 10 | 12 | 2 | 25 | Precipitate | — |
| | | | | | | forms - | |
| | | | | | | clogged | |
| | | | | | | tubing | |
| 6^{c} | 0.5 | 10 | 12 | 2 | 25 | Precipitate | 90 |
| | | | | | | forms - | |
| | | | | | | sonication | |
| | | | | | | used | |
| 7^{c} | 0.75 | 10 | 12 | 2 | 25 | Precipitate | 91 |
| | | | | | | forms - | |
| | | | | | | sonication | |
| | | | | | | used | |
| 8^{d} | 0.75 | 10 | 12 | 2 | 60 | Precipitate | — |
| | | | | | | forms over | |
| | | | | | | time | |
| 9^{e} | 0.75 | 10 | 12 | 4 | 60 | No | 90 |
| | | | | | | precipitate | |

^{a}At low diester concentrations, no precipitation of product occurred in the tubing; however, a rotary evaporator was required to isolate the product.
^{b}Product precipitated in the tubing immediately after the backpressure regulator, leading to clogging.
^{c}Tubing after backpressure regulator was submerged in a sonicating bath, which prevented clogging for occurring in tubing.
^{d}A higher Tₒᵤₜₗₑₜ was evaluated as an alternative to sonication to slow rate of cooling; precipitation occurred over time, which led to clogging.
^{e}Increasing flow rate of the system prevented any precipitation from occurring.

This result left two possible strategies to explore: 1) reconfigure the flow system so that it could sustain precipitation in flow to avoid clogging, or 2) delay the precipitation of the product so that it would only occur in the collection flask, not in the tubing.

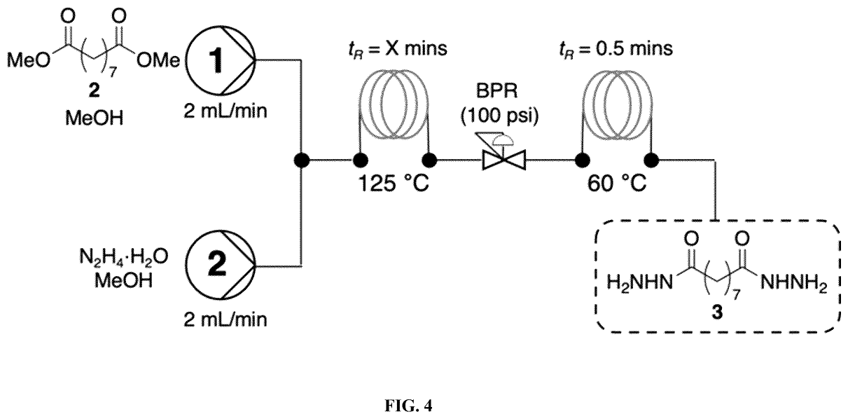

Several groups have addressed precipitation in continuous flow reactors, using a variety of different techniques, including acoustic irradiation (sonication), agitated cell reactors, or through modifying flow rates and/or tubing. Several modifications to the flow system were made (FIG. 4). All reactions were run on a 5 g scale of azelaic acid (1). First, the amount of hydrazine hydrate equivalents and the residence time were altered to identify favorable parameters, the results of which are shown in Table 2. It was also found that submerging the length of tubing which exited the backpressure regulator into a sonicating bath, coupled with an increase in tubing internal diameter, effectively prevented blockages from occurring. Alternatively, the use of a second heated coil reactor in lieu of the sonicating bath proved very efficient at avoiding uncontrolled precipitation of 3 by permitting a controlled and stepwise temperature decrease from 125° C. to 60° C. to room temperature. In conjunction with an increase in total flow rate of the system, these conditions afforded 3 in a 90% isolated yield with a very short t_{R} of 7.1 minutes (Table 2). In addition, it was found that the equivalents of hydrazine hydrate could be lowered from 10 eq. to 6.4 eq., equating to approximately 2 eq. of N₂H₄ per ester (Table 2, entry 6).

TABLE 2
Results from Optimization of the Continuous Flow Hydrazinolysis.

| | Equiv. | Diester | t_{R} Reactor | Yield of |
|---|---|---|---|---|
| Entry | N₂H₄•H₂O | Conc. (M) | (mins) | 3 (%) |
| 1 | 10 | 1.5 | 12 | 91 |
| 2 | 10 | 1.5 | 6.6 | 91 |
| 3 | 8 | 1.5 | 6.6 | 90 |
| 4 | 7 | 1.5 | 6.6 | 90 |
| 5 | 6 | 1.5 | 6.6 | 81^{b} |
| 6 | 6.4 | 1.5 | 6.6 | 90 |

Reactions were run on a 5 g scale of 1, isolated yields are reported for each entry.
^{b}Mixture of 3 and monohydrazide obtained.

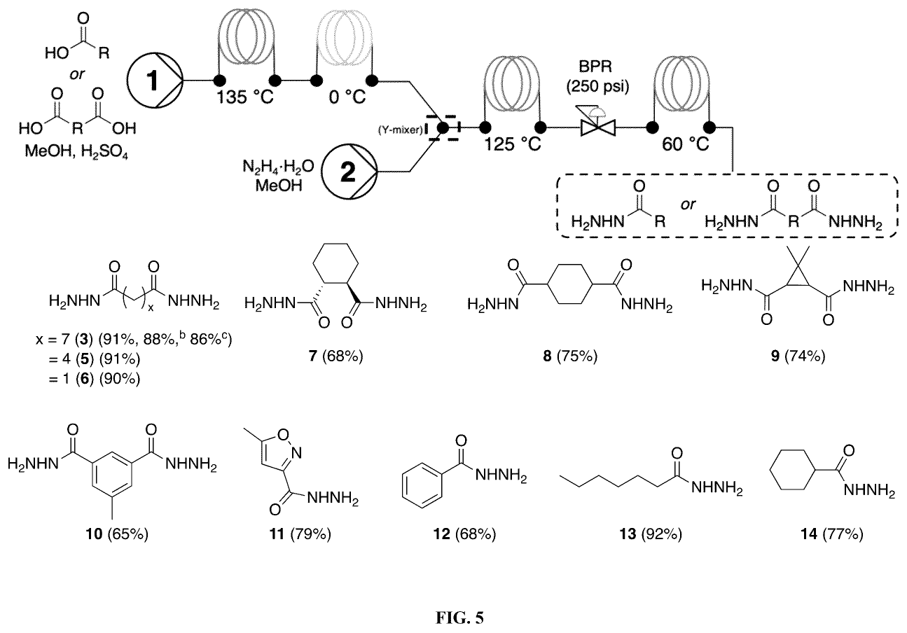

With an optimized set of hydrazinolysis conditions, the focus move to telescope the sequence (FIG. 5). Evaluating under batch conditions initially, simply mixing the crude esterification reaction stream with a methanolic solution of hydrazine hydrate afforded 3 in a 91% isolated yield. Adapting this to flow, however, small amounts of white precipitate was observed forming at the T-mixer connecting the streams from pumps 1 and 2 during the course of the reaction, which is believed to be hydrazine sulfate formation due to the catalytic sulfuric acid from the esterification step. To avoid potential clogging, a Y-mixer junction was used in all subsequent reactions, with no further precipitation issues being observed.

To investigate the generality of this methodology, a series of both aliphatic and aromatic mono/dicarboxylic acids were evaluated (FIG. 5). It was found that these conditions provide amenable to a variety of aliphatic, aromatic, and heteroaromatic functionalities to provide a broad range of mono- and dihydrazides in good to excellent yields. Isolate yields were reported in all cases. The b reaction was conducted using algae-derived azelaic acid on a 5 g scale. The c reaction was conducted on a 200 g scale of azelaic acid. In general, it was found that primary aliphatic carboxylic acids afforded the highest yields, while secondary aliphatic and aromatic carboxylic acids were less susceptible to hydrazinolysis under these conditions. Without being bound by theory, it is suspected that this is the direct result of steric effects that can influence both the esterification and hydrazinolysis reaction rates and lead to lower yields. However, these results indicate that parameter variations could overcome this (i.e., higher reaction temperature, longer t_{R}). Dihydrizides 7-10 hold particular promise as key starting materials for the preparation of previously inaccessible diisocyanates using this existing flow methodology, which could provide access to novel polyurethanes with a wide range of material properties.

To demonstrate the scalability of this process, a 200-gram scale reaction using 1 was conducted, providing 3 in 86% yield over a 9-hour continuous run using standard, lab scale flow chemistry equipment. (This production rate equates to ˜1 kg/week). As a proof of concept, algae-derived 1 was used to synthesize dihydrazide 3 in an equivalent yield and purity. Moreover, as an extension of this methodology, isoxazole hydrazide 11 was used to synthesize monoamine oxidase inhibitor Isocarboxazid (Marplan®), a clinically prescribed antidepressant.

Lastly, the recyclability of the collected filtrate solution was evaluated in subsequent runs using 1 as a model substrate. It was found that this solution could be readily recycled with little to no change in the isolated yield of 3 being observed even after three consecutive runs. Given the long-term goal of producing 3 on industrial scales, the recyclability of solvents and reagents during this process would prove extremely beneficial from both an economic and environmental aspect.

In summary, a methodology for the synthesis of acid hydrazides from their corresponding carboxylic acids has been developed using a continuous flow process with short residence times between 13 and 25 minutes. Moreover, by optimizing reagent concentration, flow rates, and reaction temperatures, it was demonstrated that precipitation of the generated acid hydrazides could be controlled. The generality of the methodology was illustrated through the synthesis of a small library of aliphatic, aromatic, and heteroaromatic acid hydrazides in yields of 65-91%. As an application, it was further demonstrated that the scaled production of 3 (200 g) through one continuous run over 9 hours, equating to an output of ˜22 g/hour using a lab-scale flow system. Taken together, this work demonstrates the efficiency of acid hydrazides with promising industrial application.

### Experimental Methods

General Experimental: Chemical reagents were purchased from Sigma Aldrich, Fischer Scientific, TCI Chemicals, Alfa Aeser or Accela. All chemicals were reagent grade and were used as is without further purification. Deuterated solvents (CDCl₃ and d₆-DMSO) were purchased from Cambridge Isotope Laboratories. ¹H NMR and ¹³C NMR spectra were recorded on a Bruker AVA 300 or JOEL ECA 400 MHz spectrometer. FT-IR was recorded using a Thermo Scientific Nicolet™ iS20 spectrometer (24 scans taken for each sample). Low-Resolution Mass Spectrometry (LRMS-ESI) was conducted on a Thermo LCQdeca spectrometer, while High-Resolution Mass Spectrometry was conducted using a Thermo Scientific LTQ Orbitrap XL mass spectrometer.

High-pressure HPLC pumps equipped with 10 mL/min pump heads and the heated coil reactor were purchased from Uniqsis. Backpressure regulators were purchases from IDEX Health and Science Technologies. Reactors were constructed from polytetrafluoroethylene (PTFE) tubing with either a 1/16″ outer diameter and 1/32″ inner diameter, or ⅛″ outer diameter and 1/16″ inner diameter with complementary PEEK fittings. The system was controlled using Uniqsis FlowControl™ software.

### Optimization of Flow Chemistry Conditions

### Esterification of Azelaic Acid in Flow:

Reaction screen using Amberlyst 36 ion-exchange resin: A glass Diba OmniFit column was packed with 8.6 g of Amberlyst 36 which occupied a 10.6 mL volume. A solution of azelaic acid (20 g, 106 mmol) in methanol (62.4 mL) was pumped through the heated packed bed reactor set to 120° C. at flow rate of 1.2 mL/min for a t_{R} of 10.6 minutes. Approximately 17 mL of solution was collected throughout the run. This solution was then extracted twice with hexanes and concentrated to afford a crude white paste which was further extracted into hexanes to afford 0.39 g (1.8 mmol) of azelaic diester in an 8% yield.

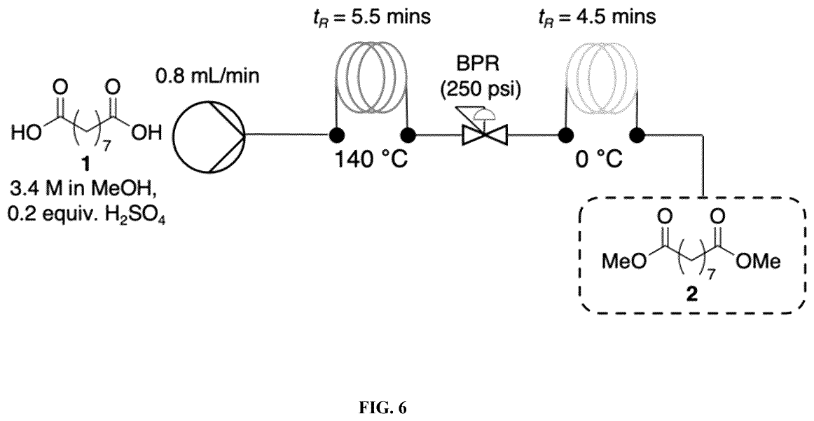

Optimized esterification reaction conditions: Flow System Operation Scheme for FIG. 6—The flow system was comprised of one pump which delivered a 3.4 M solution of 1 (azelaic acid) (5.0 g, 0.0265 mol) in acidic methanol (H₂SO₄, 0.2 equiv., 0.29 mL, 0.006 mol) at a flow rate of 0.8 mL/min (FIG. 6). The solution was pumped through a heated tubular reactor set to 140° C. for a residence time of 5.5 mins. After flowing through a 250 psi backpressure regulator, the solution was cooled in an ice bath for a residence of 4.5 mins, and then collected in an Erlenmeyer flask. The methanolic solution was then extracted three times with 10 mL of hexanes, dried over MgSO₄, filtered, and concentrated down to afford 2 (dimethyl azelate) (5.33 g, 0.0244 mol) as pale yellow oil in a 93% yield.

### Hydrazinolysis of Dimethyl Azelate Inflow:

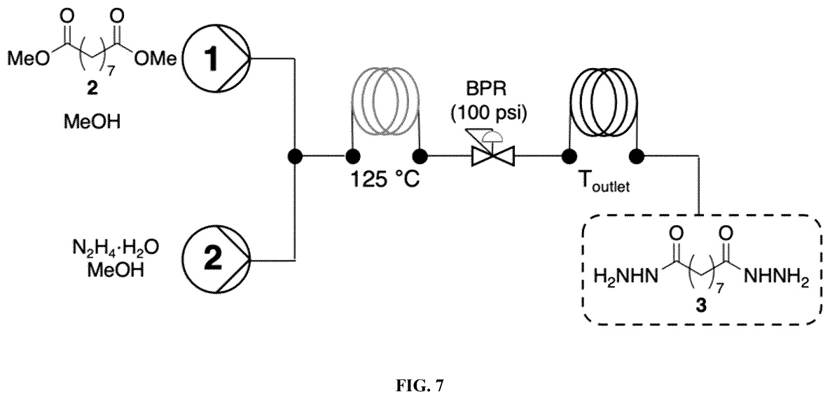

General Procedure for Reaction Optimization for FIG. 7: Pump 1 delivered a solution of 2 (5.0 g, 23.1 mmol) in methanol, while Pump 2 delivered a solution of hydrazine hydrate (10 equiv.) in methanol (FIG. 7). The streams met at a T-mixer junction and flowed through a heated coil reactor set to 125° C. After passing through a 100 psi backpressure regulator, the reaction stream then flowed through an additional segment of tubing before being collected into a flask.

In the initial runs a Tₒᵤₜₗₑₜ of 0° C. was used to rapidly cool the solution and prevent vaporization of the methanol exiting the backpressure regulator at elevated temperatures. At low diester concentrations, no precipitation occurred in the tubing (Table 1, entries 1 and 2); however, a rotary evaporator was required to isolate the product. Increasing diester concentration led to precipitation in the tubing immediately after the backpressure regulator (Table Si, entries 3 and 4)—even when the Tₒᵤₜₗₑₜ was increased to 25° C. (Table 1, entry 4). It was found that submerging the tubing after the backpressure regulator into a sonicating bath worked very well to prevent blockage and clogging from occurring—even at higher diester concentrations (Table 1, entries 6 and 7). Additionally, it was found that by increasing tubing internal diameters from 1/32″ to 1/16″, blockage in the tubing could be mitigated. Although efficient on a small lab scale, the use of a sonicating bath for the pilot scale production of azelaic dihydrazide is not ideal. As an alternative strategy, it was hypothesized that having a higher Tₒᵤₜₗₑₜ after the backpressure regulator would permit a controlled and stepwise temperature decrease from 125° C. to 60° C. to room temperature and avoid rapid precipitation from occurring in the tubing (Table 1, entry 8). While precipitation of the product occurred over time throughout this run, by increasing the flow rate of the system to 4 mL/min in a subsequent run afforded 3 in a 91% yield without any blockage in the tubing (Table 1, entry 9).

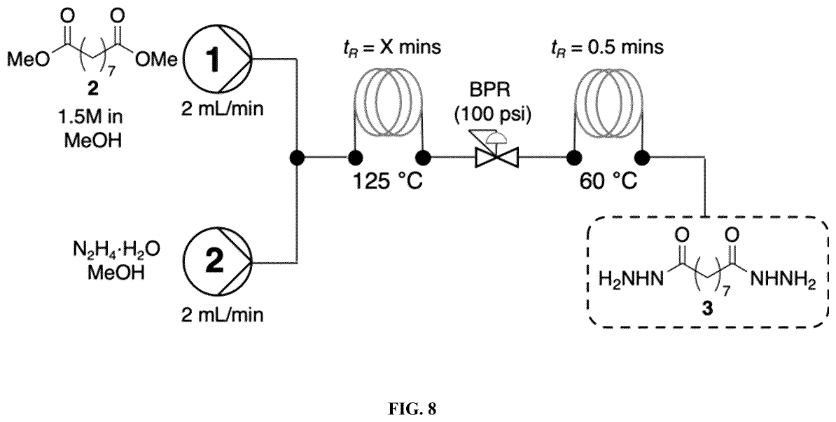

General Procedure for Reaction Optimization in FIG. 8 The general procedure for the flow system operation was identical to what was described above, and the low diagram is depicted in FIG. 8. Here, the aim was to evaluate the reaction under different residence times (t_{R}) while also optimizing the equivalents of hydrazine hydrate in an effort to reduce both (Table 3). It was found that a t_{R} of 6.6 minutes was sufficient to achieve a 91% yield of the dihydrazide (Table 3, entry 2), while decreasing the equivalents of hydrazine hydrate below 6 led to a reduction in yield as a mixture of products were obtained (Table 3, entries 5-7). In entry 8 of Table 3, the optimal conditions were found using 6.4 equivalents of hydrazine hydrate.

TABLE 3
Results of Hydrazinolysis Reaction Screen Using
Different Equivalents of Hydrazine Hydrate

| | Mass | | | | |
|---|---|---|---|---|---|
| | Azelaic | Equiv. | t_{R} Reactor | Yield of | |
| Entry | Acid (g) | N₂H₄•H₂O | (min) | 3 (%) | Result |
| 1 | 5 | 10 | 12 | 91 | Dihdrazide |
| | | | | | obtained |
| 2 | 5 | 10 | 6.6 | 91 | Dihydrazide |
| | | | | | obtained |
| 3 | 5 | 8 | 6.6 | 90 | Dihydrazide |
| | | | | | obtained |
| 4 | 5 | 7 | 6.6 | 90 | Dihydrazide |
| | | | | | obtained |
| 5 | 5 | 6 | 6.6 | 81 | Mixture of very |
| | | | | | small amounts |
| | | | | | of |
| | | | | | monohydrazide, |
| | | | | | dihydrazide |
| 6 | 5 | 5 | 6.6 | 71 | Mixture of |
| | | | | | monohydrazide, |
| | | | | | dihydrazide |
| 7 | 5 | 4 | 6.6 | 64 | Mixture of |
| | | | | | unreacted |
| | | | | | diester, |
| | | | | | monohydrazide, |
| | | | | | dihydrazide |
| 8 | 5 | 6.4 | 6.6 | 90 | Dihydrazide |
| | | | | | obtained |

### Telescoped Synthesis of Acid Hydrazides in Continuous Flow:

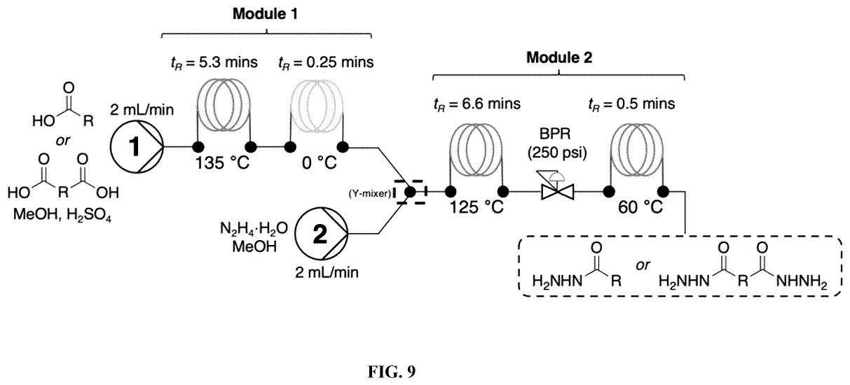

General Experimental Procedure A: An exemplary flow system may have a first pump, a first reactor, an ice bath, a second pump, a second reactor, a water bath, and a collection apparatus. More specifically, an exemplary flow system comprises of two pumps and two modules. Module 1 consists of Pump 1 which delivers a solution of carboxylic acid in acidic methanol at a flow rate of 2 mL/min to a heated coil reactor set to 135° C. with a residence time (t_{R}) of 5.3 minutes (FIG. 9). This solution then enters an ice bath for a t_{R} of 0.25 minutes to rapidly cool down the crude esterification reaction stream before entering Module 2. In Module 2, Pump 2 delivers a solution of hydrazine hydrate in methanol at a flow rate of 2 mL/min to the crude esterification reaction stream via a Y-mixer junction. These combined streams then flow at a total flow rate of 4 mL/min through a heater reactor coil set to 125° C. for a t_{R} of 6.6 minutes. After passing through a 250 psi backpressure regulator, the solution flows through a heated reactor coil set to 60° C. for a t_{R} of 0.5 minutes, and is then collected in a flask. After completion of the run, it was found that the acyl hydrazide products could either be precipitated out immediately by cooling the solution in a −78° C. bath and collected via vacuum filtration or left overnight at room temperature allowing the product to slowly precipitate out of solution and collected the following day.

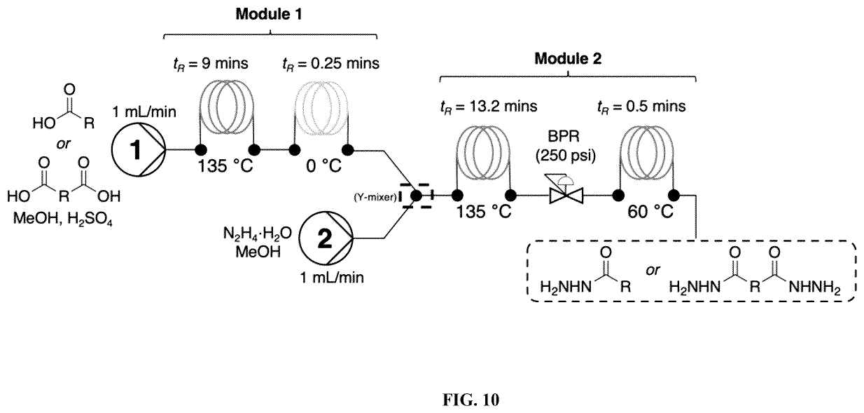

General Experimental Procedure B: For aromatic and/or secondary carboxylic acids, the flow system was slightly modified by increasing residence time and reaction temperature in Module 2. It was found that without these modifications, unreacted esters were obtained as the major product. The flow system is comprised of two pumps and two modules. Module 1 consists of Pump 1 which delivers a solution of carboxylic acid in acidic methanol at a flow rate of 1 mL/min to a heated coil reactor set to 135° C. with a residence time of (t_{R}) of 9 minutes (FIG. 10). This solution then enters an ice bath for a t_{R} of 0.25 minutes to rapidly cool down the crude esterification reaction stream before entering Module 2. In Module 2, Pump 2 delivers a solution of hydrazine hydrate in methanol at a flow rate of 1 mL/min to the crude esterification reaction stream via a Y-mixer junction. These combined streams them flow to a total flow rate of 2 mL/min through a heated reactor coil set to 135° C. for a t_{R} of 13.2 minutes. After passing through a 250 psi backpressure regulator, the solution flows through a heated reactor coil set to 60° C. for a t_{R} of 0.5 minutes, and is then collected in a flask. After completion of the run, the products were isolated as described in General Procedure A. For several of the mono-substituted acyl hydrazides, the collected reaction streams required concentration on a rotary evaporator to isolate the products owing to their solubility in methanol. Exact isolation procedures are provided for each compound in the below section.

### Substrate Scope and Compound Characterization:

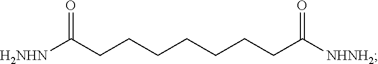

Chemical Formula: C₉H₂₀N₄O₂, Molecular Weight: 216.29.

Azelaic dihydrazide (3): The title compound was prepared according to the general flow procedure A. Pump 1 delivered a 1.5M solution of azelaic acid (5.0 g, 26.6 mmol, 1 equiv.) in methanol (18 mL) containing H₂SO₄ (0.28 mL, 5.3 mmol, 0.2 equiv.). Pump 2 delivered a 9.6M solution of N₂H₄·H₂O (8.3 mL, 170.2 mmol, 6.4 equiv.) in methanol (17.8 mL). After filtration and washing twice with methanol (15 mL), 3 was isolated as white crystals (5.2 g, 24.1 mmol) in a 91% yield. Spectral data matched those previously reported in the literature.¹¹ IR: (neat) v=3311, 3288, 2923, 1627, 1530, 980, 690 cm⁻¹. ¹H NMR (300 MHz, d₆-DMSO): δ 8.91 (s, 2H), 4.16 (s, 4H), 1.99 (t, J=7.4 Hz, 4H), 1.55-1.37 (m, 4H), 1.33-1.11 (m, 6H). ¹³C{¹H} NMR (100 MHz, d₆-DMSO): δ 172.1, 33.9, 29.1, 29.0, 25.7. LRMS (ESI) m/z: [M+H]⁺ Calcd for C₉H₂₀N₄O₂ 217.17; found 217.30.

Recycling of filtrate solution: The collected filtrate solution from the above run was concentrated down on a rotary evaporator to volume of ˜18 mL. Hydrazine hydrate (4.2 mL, 85.1 mmol, 3.2 equiv.) was then added to this solution, while 18 mL of the condensed methanol from the rotary evaporator was added to azelaic acid (5.0 g, 26.6 mmol, 1 equiv.) and H₂SO₄ (0.28 mL, 5.3 mmol, 0.2 equiv.). The flow system was operated according to the general flow procedure A, and the product was isolated as described above as white crystals (5.12 g, 23.2 mmol) in an 89% yield.

Recycling of filtrate solution (second time): The collected filtrate solution from the above run was concentrated down on a rotary evaporator to volume of ˜18 mL. Hydrazine hydrate (4.2 mL, 85.1 mmol, 3.2 equiv.) was then added to this solution, while 18 mL of the condensed methanol from the rotary evaporator was added to azelaic acid (5.0 g, 26.6 mmol, 1 equiv.) and H₂SO₄ (0.28 mL, 5.3 mmol, 0.2 equiv.). The flow system was operated according to the general flow procedure A, and the product was isolated as described above as white crystals (5.08 g, 23.2 mmol) in an 88% yield.

Synthesis of 3 using algae-derived azelaic acid: Algae-derived azelaic acid was prepared following a previously reported procedure.¹³ The procedure detailed above was identical using (5.0 g, 26.6 mmol, 1 equiv.) of algae-derived azelaic acid (5.0 g, 26.6 mmol, 1 equiv.). 3 was isolated as a white powder (5.0 g, 23.3 mmol) in an 88% yield.

Large scale synthesis of 3: Pump 1 delivered 1.3M solution of azelaic acid (200.0 g, 1.06 mol, 1 equiv.) in methanol (800 mL) containing H₂SO₄ (11.3 mL, 0.2 mol, 0.2 equiv.). Pump 2 delivered an 8.3M solution of N₂H₄·H₂O (323 mL, 6.64 mol, 6.4 equiv.) in methanol (800 mL). After filtration and washing twice with methanol (350 mL), 3 was isolated as white crystals (196.9 g, 0.91 mol) in an 86% yield.

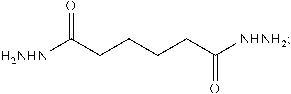

Chemical Formula: C₆H₁₄N₄O₂, Molecular Weight: 174.20

Adipic dihydrazide (5): The title compound was prepared according to the general flow procedure A. Pump 1 delivered a 1.5M solution of adipic acid (5.0 g, 34.2 mmol, 1 equiv.) in methanol (23 mL) containing H₂SO₄ (0.36 mL, 6.8 mmol, 0.2 equiv.). Pump 2 delivered a 9.6M solution of N₂H₄·H₂O (10.6 mL, 219 mmol, 6.4 equiv.) in methanol (23 mL). After filtration and washing twice with methanol (15 mL), 4 was isolated as white crystals (5.4 g, 31.1 mmol) in a 91% yield. Spectral data matched those previously reported in the literature.⁷ IR: (neat) v=3309, 3287, 3177, 2926, 2863, 1626, 1529, 1034, 984, 687 cm⁻¹. ¹H NMR (400 MHz, d₆-DMSO): δ 8.88 (s, 2H), 4.15 (s, 4H), 2.00-1.90 (m, 4H), 1.39 (m, J=7.2 Hz, 4H). ¹³C{¹H} NMR (100 MHz, d₆-DMSO): δ 172.0, 33.8, 25.5. LRMS (ESI) m/z: [M+H]⁺ Calcd for C₆H₁₅N₄O₂ 175.12; found 175.22.

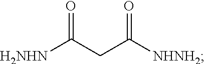

Chemical Formula: C₃H₈N₄O₂, Molecular Weight: 132.12

Malonic dihydrazide (6): The title compound was prepared according to the general flow procedure A. Pump 1 delivered a 1.5M solution of malonic acid (2.5 g, 24.0 mmol, 1 equiv.) in methanol (16 mL) containing H₂SO₄ (0.26 mL, 4.8 mmol, 0.2 equiv.). Pump 2 delivered a 9.6M solution of N₂H₄·H₂O (7.5 mL, 154 mmol, 6.4 equiv.) in methanol (16 mL). After filtration and washing twice with methanol (15 mL), 5 was isolated as white crystals (2.9 g, 21.6 mmol) in a 90% yield. Spectral data matched those previously reported in the literature.⁴⁰ IR: (neat) v=3297, 3199, 3122, 3031, 2867, 1663, 1644, 1591, 1520, 1360, 1049, 954, 692 cm⁻¹. ¹H NMR (400 MHz, d₆-DMSO): δ 9.03 (s, 2H), 4.27 (s, 4H), 2.85 (s, 2H). ¹³C{¹H} NMR (100 MHz, d₆-DMSO): δ 166.6, 38.6. LRMS (ESI) m/z: [M+H]⁺ Calcd for C₃H₉N₄O₂ 133.07; found 133.19.

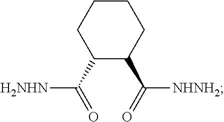

Chemical Formula: C₈H₁₆N₄O₂, Molecular Weight: 200.24.

Cyclohexane-1,2-dicarbohydrazide (7): The title compound was prepared according to the general flow procedure B. Pump 1 delivered a 0.8M solution of trans-1,2-cyclohexane dicarboxylic acid (2.5 g, 14.5 mmol, 1 equiv.) in methanol (18 mL) containing H₂SO₄ (0.15 mL, 2.9 mmol, 0.2 equiv.). Pump 2 delivered a 5.1M solution of N₂H₄·H₂O (4.5 mL, 92 mmol, 6.4 equiv.) in methanol (18 mL). After filtration and washing twice with methanol (10 mL), 6 was isolated as white crystals (1.9 g, 9.9 mmol) in a 68% yield. Spectral data matched those previously reported in the literature.⁴¹ IR: (neat) v=3238, 3170, 3068, 2938, 2856, 1626, 1549, 1275, 934, 692, 646 cm⁻¹. ¹H NMR (400 MHz, d₆-DMSO): δ 8.79 (s, 2H), 4.09 (s, 4H), 2.38-2.27 (m, 2H), 1.66 (m, J=12.0 Hz, 4H), 1.19 (m, J=30.1, 11.1 Hz, 4H). ¹³C{¹H} NMR (100 MHz, d₆-DMSO) δ 174.2, 43.9, 30.3, 25.6. LRMS (ESI) m/z: [M+H]⁺ Calcd for C₈H₁₇N₄O₂ 201.14; found 201.17.

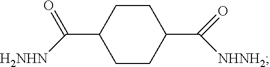

Chemical Formula: C₈H₁₆N₄O₂, Molecular Weight: 200.24.

Cyclohexane-1,4-dicarbohydrazide (8): The title compound was prepared according to the general flow procedure B. Pump 1 delivered a 1.45M solution of 1,4-cyclohexane dicarboxylic acid (2.5 g, 14.5 mmol, 1 equiv.) in methanol (10 mL) containing H₂SO₄ (0.15 mL, 2.9 mmol, 0.2 equiv.). Pump 2 delivered a 9.3M solution of N₂H₄·H₂O (4.6 mL, 93 mmol, 6.4 equiv.) in methanol (10 mL). After filtration and washing twice with methanol (10 mL), a white solid was obtained. A recrystallization in water then afforded 7 as white crystals (2.1 g, 10.9 mmol) in a 75% yield. Spectral data matched those previously reported in the literature.⁴² IR: (neat) v=3297, 3188, 2935, 2859, 1625, 1532, 1266, 965, 637 cm⁻¹. ¹H NMR (400 MHz, d₆-DMSO): δ 8.87 (s, 2H), 4.08 (s, 4H), 1.97 (m, 2H), 1.64 (m, J=8.2 Hz, 4H), 1.32 (m, J=10.2 Hz, 4H). ¹³C{¹H} NMR (100 MHz, d₆-DMSO): δ 174.9, 42.1, 28.8. LRMS (ESI) m/z: [M+H]⁺ Calcd for CH₁₇N₄O₂ 201.14; found 201.28.

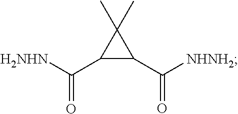

Chemical Formula: C₇H₁₄N₄O₂, Molecular Weight: 186.22.

3,3-dimethylcyclopropane-1,2-dicarbohydrazide (9): The title compound was prepared according to the general flow procedure B. Pump 1 delivered a 1.5M solution of 3,3-dimethylcyclopropane-1,2-dicarboxylic acid¹ (1.0 g, 6.3 mmol, 1 equiv.) in methanol (4.2 mL) containing H₂SO₄ (0.07 mL, 1.3 mmol, 0.2 equiv.). Pump 2 delivered a 9.6M solution of N₂H₄·H₂O (2.3 mL, 48 mmol, 6.4 equiv.) in methanol (5 mL). After filtration and washing twice with cold methanol (10 mL), 8 was isolated as a white powder (0.87 g, 4.7 mmol) in a 74% yield. Spectral data matched those previously reported in the literature.¹¹ IR: (neat) v=3292, 3182, 2958, 1606, 1521, 1361, 1028, 684 cm⁻¹. ¹H NMR (400 MHz, d₆-DMSO): δ 9.12 (s, 2H), 4.14 (s, 4H), 1.92 (s, 2H), 1.11 (s, 6H). ¹³C{¹H} NMR (100 MHz, d₆-DMSO): δ 169.5, 31.6, 27.6, 20.6. LRMS (ESI) m/z: [M+H]⁺ Calcd for C₇H₁₅N₄O₂ 187.12; found 187.19.

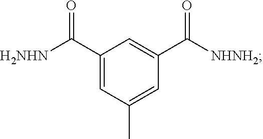

Chemical Formula: C₉H₁₂N₄O₂, Molecular Weight: 208.22.

5-methylisophthalohydrazide (10): The title compound was prepared according to the general flow procedure B. Pump 1 delivered a 0.55M solution of 5-methylisophthalic acid (2.5 g, 13.8 mmol, 1 equiv.) in methanol (25 mL) containing H₂SO₄ (0.15 mL, 2.8 mmol, 0.2 equiv.). To fully solubilize the dicarboxylic acid, the solution was gently warmed with continuous stirring at 45° C. for ten minutes, and then cooled to room temperature before being used. Pump 2 delivered a 3.55M solution of N₂H₄·H₂O (4.3 mL, 89 mmol, 6.4 equiv.) in methanol (25 mL). After filtration and washing twice with methanol (10 mL), 9 was isolated as white powder (1.9 g, 9.0 mmol) in a 65% yield. Spectral data matched those previously reported in the literature.¹¹ IR: (neat) v=3291, 3177, 2923, 1636, 1599, 1512, 1309, 679, cm⁻¹. ¹H NMR (400 MHz, d₆-DMSO): δ 9.70 (s, 2H), 7.99 (s, 1H), 7.70 (s, 2H), 4.54 (s, 4H), 2.33 (s, 3H). ¹³C{¹H} NMR (100 MHz, d₆-DMSO): δ 166.1, 138.4, 134.1, 130.5, 123.7, 21.4. LRMS (ESI) m/z: [M+H]⁺ Calcd for C₉H₁₂N₄O₂ 209.11; found 209.25.

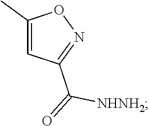

Chemical Formula: C₅H₇N₃O₂, Molecular Weight: 141.13

5-methylisoxazole-3-carbohydrazide (11): The title compound was prepared according to the general flow procedure B. Pump 1 delivered a 0.8M solution of 5-methylisoxazole-3-carboxlyic acid (2.0 g, 15.7 mmol, 1 equiv.) in methanol (20 mL) containing H₂SO₄ (0.17 mL, 3.1 mmol, 0.2 equiv.). To fully solubilize the carboxylic acid, the solution was gently warmed with continuous stirring at 45° C. for ten minutes, and then cooled to room temperature before being used. Pump 2 delivered a 2.6M solution of N₂H₄·H₂O (2.5 mL, 52 mmol, 3.2 equiv.) in methanol (20 mL). After completion of the run, the collected solution was concentrated on a rotary evaporator to afford a crude white paste. The crude mixture was then washed with cold methanol (10 mL) and then cold water (10 mL) to provide 10 as an off-white powder (1.75 g, 12.4 mmol) in a 79% yield. Spectral data matched those previously reported in the literature.⁴³ IR: (neat) v=3309, 3234, 1659, 1648, 1608, 1553, 1458, 1234, 917, 810, 709 cm⁻¹. ¹H NMR (400 MHz, d₆-DMSO): δ 9.90 (s, 1H), 6.48 (s, 1H), 4.54 (s, 2H), 2.40 (s, 3H). ¹³C {¹H} NMR (75 MHz, d₆-DMSO): δ 171.3, 158.5, 158.5, 101.5, 12.2. LRMS (ESI) m/z: [M+H]⁺ Calcd for C₅H₈N₃O₂ 142.06; found 142.19.

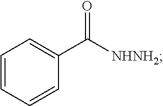

Chemical Formula: C₇H₈N₂O, Molecular Weight: 136.15.

Benzhydrazide (12): The title compound was prepared according to the general flow procedure B. Pump 1 delivered a 1.5M solution of benzoic acid (2.5 g, 20.5 mmol, 1 equiv.) in methanol (14 mL) containing H₂SO₄ (0.22 mL, 4.1 mmol, 0.2 equiv.). Pump 2 delivered a 4.8M solution of N₂H₄·H₂O (3.3 mL, 67.2 mmol, 3.2 equiv.) in methanol (14 mL). After completion of the run, the collected solution was concentrated on a rotary evaporator to afford a crude white paste. The crude mixture was then dissolved in 10 mL of 2-propanol, and the product was precipitated out by adding 100 mL of cold hexanes. After filtration, 11 was isolated as white crystals (1.9 g, 13.9 mmol) in a 68% yield. Spectral data matched those previously reported in the literature.¹¹ IR: (neat) v=3298, 3195, 2999, 2874, 1613, 1559, 1344, 986, 682, 670 cm⁻¹. ¹H NMR (400 MHz, d₆-DMSO): δ 9.73 (s, 1H), 7.88-7.70 (m, 2H), 7.50-7.44 (m, 1H), 7.44-7.37 (m, 2H), 4.49 (s, 2H). ¹³C{¹H} NMR (100 MHz, d₆-DMSO): δ 166.4, 133.8, 131.6, 128.9, 127.5. LRMS (ESI) m/z: [M+H]⁺ Calcd for C₇H₉N₂O 136.07; found 136.21.

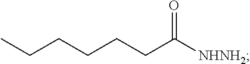

Chemical Formula: C₇H₁₆N₂O, Molecular Weight: 144.22.

Heptane hydrazide (13): The title compound was prepared according to the general flow procedure A. Pump 1 delivered a 1.5M solution of heptanoic acid (5.0 g, 38.4 mmol, 1 equiv.) in methanol (25.6 mL) containing H₂SO₄ (0.41 mL, 7.7 mmol, 0.2 equiv.). Pump 2 delivered a 4.8M solution of N₂H₄·H₂O (5.8 mL, 120 mmol, 3.2 equiv.) in methanol (25 mL). After filtration and washing twice with cold methanol (15 mL), 12 was isolated as white needle-like crystals (5.1 g, 35.3 mmol) in a 92% yield. Spectral data matched those previously reported in the literature.¹¹ IR: (neat) v=3293, 3197, 2927, 2850, 1624, 1530, 979, 645 cm⁻¹. ¹H NMR (400 MHz, d₆-DMSO): δ 8.87 (s, 1H), 4.08 (s, 2H), 1.95 (t, J=7.4 Hz, 2H), 1.43 (m, J=7.4 Hz, 2H), 1.19 (m, J=11.8 Hz, 6H), 0.80 (t, J=7.1 Hz, 3H). ¹³C{¹H} NMR (100 MHz, d₆-DMSO): δ 172.2, 33.9, 31.5, 28.9, 25.7, 22.5, 14.5. LRMS (ESI) m/z: [M+H]⁺ Calcd for C₇H₁₇N₂O 145.14; found 145.27.

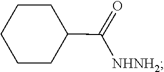

Chemical Formula: C₇H₁₄N₂O, Molecular Weight: 142.20.

Cyclohexane hydrazide (14): The title compound was prepared according to the general flow procedure A. Pump 1 delivered a 1.6M solution of cyclohexane carboxylic acid (2.0 g, 15.6 mmol, 1 equiv.) in methanol (10.5 mL) containing H₂SO₄ (0.17 mL, 3.1 mmol, 0.2 equiv.). Pump 2 delivered a 4.9M solution of N₂H₄·H₂O (2.4 mL, 49 mmol, 3.2 equiv.) in methanol (10 mL). After completion of the run, the collected solution was concentrated on a rotary evaporator to afford a crude white paste. The crude mixture was then dissolved in 10 mL of 2-propanol, and the product was precipitated out by adding 100 mL of cold hexanes. After filtration, 13 was isolated as a white powder (1.7 g, 12.0 mmol) in a 77% yield. Spectral data matched those previously reported in the literature.¹⁸ IR: (neat) v=3297, 2928, 2850, 1623, 1530, 978, 642 cm⁻¹. ¹H NMR (300 MHz, d₆-DMSO): δ 8.87 (s, 1H), 4.13 (s, 2H), 2.03 (tt, J=11.6, 3.5 Hz, 1H), 1.77-1.55 (m, 5H), 1.44-1.10 (m, 5H). ¹³C{¹H} NMR (100 MHz, d₆-DMSO): δ 175.2, 42.8, 29.7, 25.9, 25.8. LRMS (ESI) m/z: [M+H]⁺ Calcd for C₇H₁₄N₂O 143.12; found 143.26.

### Synthesis of Isocarboxazid Using Compound 11:

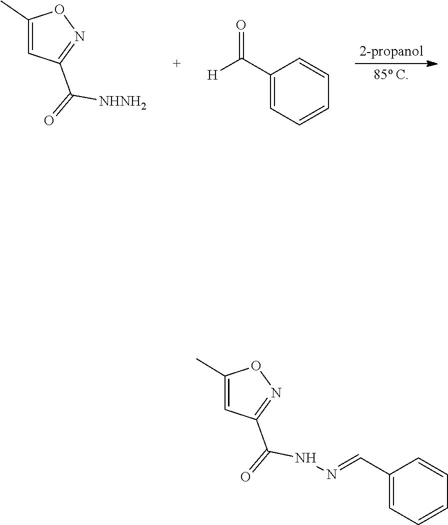

Chemical Formula: C₅H₇N₃O₂, Molecular Weight: 141.13; Chemical Formula: C₇H₆O, Molecular Weight: 106.12; Chemical Formula: C₁₂H₁₁N₃O₂, Molecular Weight: 229.24.

Benzyldiazenyl(5-methylisoxazol-3-yl)methanone (15): To a solution of benzaldehyde (537 mg, 5.3 mmol, 1.5 equiv.) in 2-propanol (15 mL), was added 10 (500 mg, 3.5 mmol, 1 equiv.), and the mixture was heated to 85° C. for one hour. The mixture was then cooled in an ice bath with stirring for 30 minutes. A white precipitate formed and was collected via vacuum filtration and washed with cold 2-propanol (10 mL) to afford 14 as a white solid (755 mg, 3.3 mmol) in a 94% yield. IR: (neat) v=3277, 3140, 2928, 2855, 2362, 1722, 1674, 1542, 1263, 833, 758, 721 cm⁻¹. ¹H NMR (400 MHz, CDCl₃) δ 9.75 (s, 1H), 8.20 (s, 1H), 7.83-7.74 (m, 2H), 7.46-7.37 (m, 3H), 6.58 (s, 1H), 2.52 (s, 3H). ¹³C{¹H} NMR (100 MHz, d₆-DMSO) δ 172.0, 158.7, 155.9, 150.0, 134.5, 131.0, 129.4, 127.8, 102.3, 12.4. HRMS (ESI) m/z: [M+H]⁺ Calcd for C₁₂H₁₂N₃O₂ 230.0294; found 230.0295.

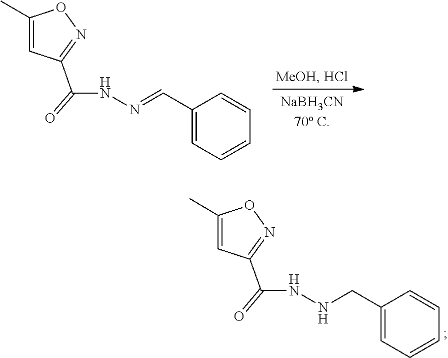 [SMILES: Cc1cc(C(=O)N/N=C/c2ccccc2)no1 ; Cc1cc(C(=O)NNCc2ccccc2)no1]

Chemical Formula: C₁₂H₁₁N₃O₂, Molecular Weight: 229.24; Chemical Formula: C₁₂H₁₃N₃O₂, Molecular Weight: 231.26.

N′-benzyl-5-methylisoxazole-3-carbohydrazide (Isocarboxazid) (16): A slightly modified procedure from Frazão de Faria was used for the reduction. Briefly, sodium cyanoborohydride (274 mg, 4.4 mmol, 2 equiv.) was added to a solution of methanol (20 mL) containing 14 (500 mg, 2.2 mmol, 1 equiv.). The mixture was carefully acidified to a pH of ˜3 by the addition of 5M methanolic HCl and then refluxed for 5 hours. After concentrating the mixture on a rotary evaporator, the crude white solid was dissolved in 25 mL DI water and the pH was adjusted to >7 by the addition of 6M aqueous NaOH which caused a white precipitate to form. The white precipitate was collected via vacuum filtration and washed with DI water (20 mL) to afford 15 as a white solid (310 mg, 1.34 mmol) in a 62% yield. IR: (neat) v=3278, 3212, 3029, 2884, 1673, 1456, 810, 745, 702 cm⁻¹. ¹H NMR (400 MHz, d6-DMSO) δ 10.15 (s, 1H), 7.35-7.19 (m, 5H), 6.46 (d, J=1.0 Hz, 1H), 5.49 (t, J=5.6 Hz, 1H), 3.93 (d, J=5.3 Hz, 2H), 2.40 (d, J=0.9 Hz, 3H). ¹³C{¹H} NMR (100 MHz, d6-DMSO) δ 171.5, 158.5, 158.3, 138.8, 129.0, 128.7, 127.6, 101.7, 54.8, 12.3. HRMS (ESI) m/z: [M+H]⁺ Calcd for C₁₂H₁₄N₃O₂ 232.1081; found 232.1081.

### Embodiments

Embodiment 1: A continuous process for preparing a compound of Formula (III)

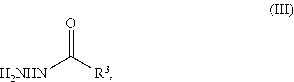

or a salt and/or solvate thereof, comprising: (a) forming a first reaction stream comprising a compound of Formula (I) dissolved in an alcohol

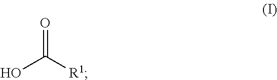

(b) flowing the reaction stream from step (a) through a continuous flow reactor at a temperature of about 100° C. to about 160° C. and at a flow rate of about 0.2 mL/min to about 5 mL/min; (c) contacting the reaction stream from step (b) with a second reaction stream comprising a hydrazine source to form a combined reaction stream; and (d) flowing the combined reaction stream from step (c) through a continuous flow reactor at a temperature of about 100° C. to about 160° C. and at a flow rate of about 0.2 mL/min to about 5 mL/min; wherein R¹ is Y or

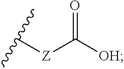

### R³ is Y or

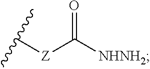

Y is an aryl, heteroaryl, alkyl, cycloalkyl, or heterocycloalkyl group; Z is a divalent group selected from arylene, heteroarylene, alkylene, cycloalkylene, and heterocycloalkylene groups; and R¹ and R³ are both Y, or R¹ and R³ are both not Y.

Embodiment 2: The continuous process of Embodiment 1, wherein the alcohol in step (a) has a formula of R^{a}OH, wherein R^{a} is a C₁-C₆ alkyl.

Embodiment 3: The continuous process of Embodiment 2, wherein the compound of Formula (I) is converted to a compound of Formula (II) in step (b),

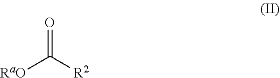

wherein: R² is Y or

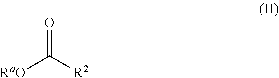

and when R¹ and R³ are both Y, R² is Y; when R¹ and R³ are both not Y, R² is not Y.

Embodiment 4: The continuous process of any one of Embodiments 1-3, wherein the first reaction stream further comprises an acid, and wherein the acid is present in an amount of less than 0.5 equivalent relative to the amount of a compound of Formula (I).

Embodiment 5: The continuous process of any one of Embodiments 1-4, wherein the acid in the first reaction stream is H₂SO₄.

Embodiment 6: The continuous process of any one of Embodiments 1-5, wherein the compound of Formula (I) has a concentration of about 0.2 M to about 2 M in the first reaction stream.

Embodiment 7: The continuous process of any one of Embodiments 1-6, wherein the temperature in step (b) is about 135° C.

Embodiment 8: The continuous process of any one of Embodiments 1-7, wherein the flow rate in step (b) is about 2 mL/min or about 1 mL/min.

Embodiment 9: The continuous process of any one of Embodiments 1-8, wherein the flow rate in step (b) provides a residence time of about 2 minutes to about 15 minutes.

Embodiment 10: The continuous process of any one of Embodiments 1-9, wherein the flow rate in step (b) provides a residence time of about 5.3 minutes or about 9 minutes.

Embodiment 11: The continuous process of any one of Embodiments 1-10, further comprising cooling the reaction stream from step (b) to about 0° C. before step (c).

Embodiment 12: The continuous process of Embodiment 11, wherein the cooling step comprises flowing the reaction stream from step (b) through an ice bath with a residence time of less than about 1 minute, or less than about 0.5 minutes.

Embodiment 13: The continuous process of Embodiment 12, wherein the residence time in the cooling step is about 0.25 minutes.

Embodiment 14: The continuous process of any one of Embodiments 1-13, wherein the second reaction stream in step (c) comprises an alcohol solvent.

Embodiment 15: The continuous process of any one of Embodiments 1-14, wherein the alcohol solvent in step (c) is methanol.

Embodiment 16: The continuous process of any one of Embodiments 1-15, wherein the hydrazine source in step (c) is hydrazine hydrate.

Embodiment 17: The continuous process of any one of Embodiments 1-16, wherein the hydrazine source is present in the second reaction stream in an amount of about 2 equivalents to about 5 equivalents relative to the amount of —COOH functional group in the compound of Formula (I).

Embodiment 18: The continuous process of any one of Embodiments 1-17, wherein the hydrazine source has a concentration of about 2 M to about 10 M.

Embodiment 19: The continuous process of any one of Embodiments 1-18, wherein the temperature in step (d) is about 125° C. or about 135° C.

Embodiment 20: The continuous process of any one of Embodiment 1-19, wherein the flow rate in step (d) is about 2 mL/min or about 1 mL/min.

Embodiment 21: The continuous process of any one of Embodiments 1-20, wherein the flow rate in step (d) provides a residence time of about 2 minutes to about 15 minutes.

Embodiment 22: The continuous process of any one of Embodiments 1-21, wherein the flow rate in step (d) provide a residence time of about 6.6 minutes or about 13.2 minutes.

Embodiment 23: The continuous process of any one of Embodiments 1-22, further comprising (e) flowing the reaction stream from step (d) through a backpressure regulator; and (f) cooling the reaction stream from step (e) to about 60° C.

Embodiment 24: The continuous process of Embodiment 23, wherein the backpressure regulator in step (e) is a 250 psi backpressure regulator.

Embodiment 25: The continuous process of Embodiment 23, wherein the cooling step comprises flowing the reaction stream from step (e) through a continuous flow reactor at a temperature of about 60° C., and with a residence time of less than about 1 minute.

Embodiment 26: The continuous process of any one of Embodiments 23-25, wherein the residence time in the cooling step (f) is about 0.5 minutes.

Embodiment 27: The continuous process of any one of Embodiments 1-26, further comprising collecting a reactor effluent exiting from the continuous flow reactor.

Embodiment 28: The continuous process of Embodiment 27, further comprising cooling the reactor effluent exiting from the continuous flow reactor, thereby forming precipitates of a compound of Formula (III).

Embodiment 29: The continuous process of any one of Embodiments 1-28, further comprising isolating the compound of Formula (III).

Embodiment 30: The continuous process of Embodiment 29, further comprising collecting any remaining material after the compound of Formula (III) is isolated, and repeating step (a) using said remaining material.

Embodiment 31: The continuous process of any one of Embodiments 1-30, wherein the compound of Formula (I) is a compound having a formula of

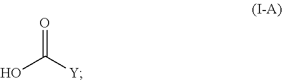

and
the compound of Formula (III) is a compound having a formula of

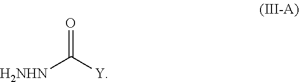

Embodiment 32: The continuous process of Embodiment 31, wherein the compound of Formula (I-A) is converted to a compound of Formula (II-A) in step (b),

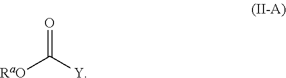

Embodiment 33: The continuous process of any one of Embodiments 1-32, wherein Y is selected from the group consisting of isoxazolyl, phenyl, alkyl, and cycloalkyl.

Embodiment 34: The continuous process of any one of Embodiments 1-33, wherein Y is selected from the group consisting of

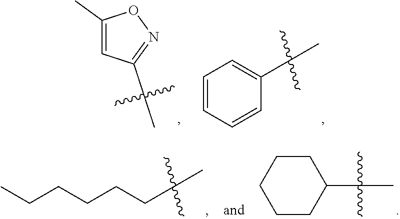

Embodiment 35: The continuous process of any one of Embodiments 1-30, wherein the compound of Formula (I) is a compound having the formula of

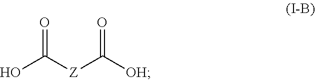

and
the compound of Formula (III) is a compound having a formula of

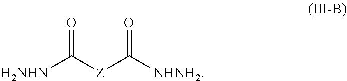

Embodiment 36: The continuous process of Embodiment 35, wherein the compound of Formula (I-B) is converted to a compound of Formula (II-B) in step (b),

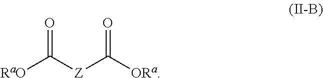

Embodiment 37: The continuous process of any one of Embodiments 1-30 and 35-36, wherein Z is selected from the group consisting of phenylene, alkylene, and cycloalkylene.

Embodiment 38: The continuous process of any one of Embodiments 1-30 and 35-37 wherein Z is selected from the group consisting of

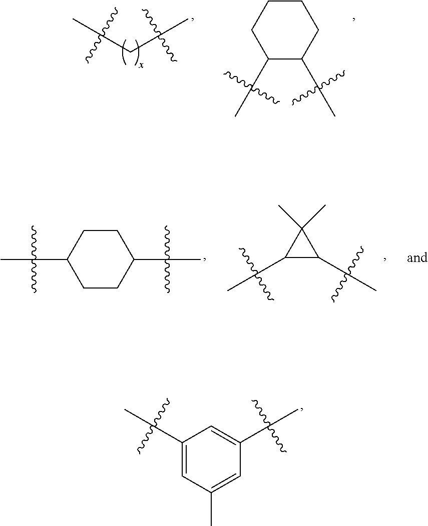

wherein x is an integer from 1-7.

Embodiment 39: The continuous process of any one of Embodiments 1-38, wherein R^{a} is methyl.

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entireties, to the same extent as if each were incorporated by reference individually. In case of conflict, the present specification, including definitions, will control.

Other aspects are set forth in the claims.

### REFERENCES

1. Aggarwal, N.; Kumar, R.; Srivastva, C.; Dureja, P.; Khurana, J. M., Synthesis of Nalidixic Acid Based Hydrazones as Novel Pesticides. J. Agric. Food Chem. 2010, 58 (5), 3056-3061.
2. Gomes, D.; Pinto, J. C.; Borges, C., Determination of hydrazide content in poly(oxadiazole-hydrazide) copolymers by NMR and thermal analysis. Polymer 2003, 44 (20), 6223-6233.
3. Majumdar, P.; Pati, A.; Patra, M.; Behera, R. K.; Behera, A. K., Acid Hydrazides, Potent Reagents for Synthesis of Oxygen-, Nitrogen-, and/or Sulfur-Containing Heterocyclic Rings. Chem. Rev. 2014, 114 (5), 2942-2977.
4. Spiliopoulou, N.; Constantinou, C. T.; Triandafillidi, I.; Kokotos, C. G., Synthetic Approaches to Acyl Hydrazides and Their Use as Synthons in Organic Synthesis. Synthesis 2020, 52 (21), 3219¬3230.
5. Shamsabadi, A.; Chudasama, V., An overview of the synthesis of acyl hydrazides from aldehydes and reactions of the products thereof. Org. Biomol. Chem. 2017, 15 (1), 17-33.
6. Lee, J. H., Using Dihydrazides as Thermal Latent Curing Agents in Epoxy-Based Sealing Materials for Liquid Crystal Displays. Polymers 2021, 13 (1), 109.
7. Al-Amiery, A. A.; Binti Kassim, F. A.; Kadhum, A. A. H.; Mohamad, A. B., Synthesis and characterization of a novel eco-friendly corrosion inhibition for mild steel in 1 M hydrochloric acid. Sci. Rep. 2016, 6(1), 19890.
8. Kascholke, C.; Loth, T.; Kohn-Polster, C.; Moller, S.; Bellstedt, P.; Schulz-Siegmund, M.; Schnabelrauch, M.; Hacker, M. C., Dual-Functional Hydrazide-Reactive and Anhydride-Containing Oligomeric Hydrogel Building Blocks. Biomacromolecules 2017, 18 (3), 683-694.
9. Mata, A.; Weigl, U.; FlOgel, O.; Baur, P.; Hone, C. A.; Kappe, C. O., Acyl azide generation and amide bond formation in continuous-flow for the synthesis of peptides. React. Chem. Eng. 2020, 5 (4), 645-650.
10. Sprecher, H.; Payan, M. N. P.; Weber, M.; Yilmaz, G.; Wille, G., Acyl azide synthesis and curtius rearrangements in microstructured flow chemistry systems. J. Flow Chem. 2012, 2 (1), 20-23.
11. Phung Hai, T. A.; De Backer, L. J. S.; Cosford, N. D. P.; Burkart, M. D., Preparation of Mono- and Diisocyanates in Flow from Renewable Carboxylic Acids. Org. Process Res. Dev. 2020, 24 (10), 2342-2346.
12. Rajput, B. S.; Hai, T. A. P.; Burkart, M. D., High Bio-Content Thermoplastic Polyurethanes from Azelaic Acid. Molecules 2022, 27 (15), 4885-4885.
13. Phung Hai, T. A.; Samoylov, A. A.; Rajput, B. S.; Burkart, M. D., Laboratory Ozonolysis Using an Integrated Batch—DIY Flow System for Renewable Material Production. ACS Omega 2022, 7 (18), 15350-15358.
14. Sharba, A.; Al-Bayati, R.; Aouad, M.; Rezki, N., Synthesis of Oxadiazoles, Thiadiazoles and Triazoles Derived from Benzo[b]thiophene. Molecules 2005, 10 (9), 1161-1168.
15. Westerterp, K. R.; Molga, E. J., Safety and Runaway Prevention in Batch and Semibatch Reactors—A Review. Chem. Eng. Res. Des. 2006, 84 (7), 543-552.
16. Peng, Y.; Song, G., An Efficient Microwave-Assisted One-Pot Conversion of Carboxylic Acids into Hydrazides. J. Chem. Res. 2003, 2003 (12), 768-769.
17. Saha, A.; Kumar, R.; Kumar, R.; Devakumar, C., Development and assessment of green synthesis of hydrazides. Indian J. Chem. 2010, 49B, 526-531.
18. Zhang, X.; Breslav, M.; Grimm, J.; Guan, K.; Huang, A.; Liu, F.; Maryanoff, C. A.; Palmer, D.; Patel, M.; Qian, Y.; Shaw, C.; Sorgi, K.; Stefanick, S.; Xu, D., A New Procedure for Preparation of Carboxylic Acid Hydrazides. J Org. Chem. 2002, 67 (26), 9471-9474.
19. Antonio De La, H.; Jesús, A.; José, C.; María, A. H.; Juan De, M. M.; Pilar, P.; Abel De, C.; Angel, D.-O., Reproducibility and Scalability of Microwave-Assisted Reactions. In Microwave Heating, Usha, C., Ed. IntechOpen: Rijeka, 2011; p Ch. 7.
20. Sharma, S.; Buchbinder, N. W.; Braje, W. M.; Handa, S., Fast Amide Couplings in Water: Extraction, Column Chromatography, and Crystallization Not Required. Org. Lett. 2020, 22 (15), 5737-5740.
21. Plutschack, M. B.; Pieber, B.; Gilmore, K.; Seeberger, P. H., The Hitchhiker's Guide to Flow Chemistry. Chem. Rev. 2017, 117(18), 11796-11893.
22. Breen, C. P.; Nambiar, A. M. K.; Jamison, T. F.; Jensen, K. F., Ready, Set, Flow!Automated Continuous Synthesis and Optimization. Trends Chem. 2021, 3 (5), 373-386.
23. Britton, J.; Raston, C. L., Multi-step continuous-flow synthesis. Chem. Soc. Rev. 2017, 46(5), 1250-1271.
24. Guidi, M.; Moon, S.; Anghileri, L.; Cambie, D.; Seeberger, P. H.; Gilmore, K., Combining radial and continuous flow synthesis to optimize and scale-up the production of medicines. React. Chem. Eng. 2021, 6 (2), 220-224.
25. Baek, H.; Minakawa, M.; Yamada, Y. M. A.; Han, J. W.; Uozumi, Y., In-Water and Neat Batch and Continuous-Flow Direct Esterification and Transesterification by a Porous Polymeric Acid Catalyst. Sci. Rep. 2016, 6(1), 25925.
26. Furuta, A.; Fukuyama, T.; Ryu, I., Efficient Flow Fischer Esterification of Carboxylic Acids with Alcohols Using Sulfonic Acid-Functionalized Silica as Supported Catalyst. Bull. Chem. Soc. Jpn. 2017, 90 (5), 607-612.
27. Hu, H.; Ota, H.; Baek, H.; Shinohara, K.; Mase, T.; Uozumi, Y.; Yamada, Y. M. A., Second-Generation meta-Phenolsulfonic Acid-Formaldehyde Resin as a Catalyst for Continuous-Flow Esterification. Org. Lett. 2020, 22 (1), 160-163.
28. Britton, J.; Dalziel, S. B.; Raston, C. L., The synthesis of di-carboxylate esters using continuous flow vortex fluidics. Green Chem. 2016, 18 (7), 2193-2200.
29. Audubert, C.; Lebel, H., Mild Esterification of Carboxylic Acids via Continuous Flow Diazotization of Amines. Org. Lett. 2017, 19 (16), 4407-4410.
30. Caputo, D.; Casiello, M.; Laurenza, A. G.; Fracassi, F.; Fusco, C.; Nacci, A.; D'Accolti, L., Preparation of Biowax Esters in Continuous Flow Conditions. ACS Omega 2019, 4 (7), 12286-12292.
31. Poolman, J. M.; Maity, C.; Boekhoven, J.; van der Mee, L.; le Sage, V. A. A.; Groenewold, G. J. M.; van Kasteren, S. I.; Versluis, F.; van Esch, J. H.; Eelkema, R., A toolbox for controlling the properties and functionalisation of hydrazone-based supramolecular hydrogels. J. Mater. Chem. B 2016, 4 (5), 852-858.
32. Sedelmeier, J.; Ley, S. V.; Baxendale, I. R.; Baumann, M., KMnO4-Mediated Oxidation as a Continuous Flow Process. Org. Lett. 2010, 12 (16), 3618-3621.
33. Browne, D. L.; Deadman, B. J.; Ashe, R.; Baxendale, I. R.; Ley, S. V., Continuous Flow Processing of Slurries: Evaluation of an Agitated Cell Reactor. Org. Process Res. Dev. 2011, 15 (3), 693-697.
34. Hartman, R. L.; Naber, J. R.; Zaborenko, N.; Buchwald, S. L.; Jensen, K. F., Overcoming the Challenges of Solid Bridging and Constriction during Pd-Catalyzed C—N Bond Formation in Microreactors. Org. Process Res. Dev. 2010, 14 (6), 1347-1357.
35. Lin, H.; Dai, C.; Jamison, T. F.; Jensen, K. F., A Rapid Total Synthesis of Ciprofloxacin Hydrochloride in Continuous Flow. Angew. Chem. Int. Ed. 2017, 56 (30), 8870-8873.
36. Kučár, Š.; Kozák, J.; Matulová, M., Partial Hydrolysis of Acyl Derivatives of Saccharides. 3. Methanolysis and Hydrazinolysis of Acyl Derivatives of α- and β-d-Xylopyranosides. Chem. Pap. 1992, 46 OA, 61-65.
37. Kucar, S.; Tvaroska, I.; Zemek, J.; Anderle, D.; Matulova, M., Partial Methanolysis And Hydrazinolysis Of Some Derivatives Of 1, 6-Anhydro-Beta-D-Glucopyranose. Chem. Pap. 1988, 42 (3), 389¬399.
38. Vardanyan, R. S.; Hruby, V. J., 7-Antidepressants. In Synthesis of Essential Drugs, Vardanyan, R. S.; Hruby, V. J., Eds. Elsevier: Amsterdam, 2006; pp 103-116.
39. Aboagye, E. A.; Chea, J. D.; Yenkie, K. M., Systems level roadmap for solvent recovery and reuse in industries. iScience 2021, 24 (10), 103114.
40. Arunachalam, R.; Aswathi, C. S.; Das, A.; Kureshy, R. I.; Subramanian, P. S., Diastereoselective Nitroaldol Reaction Catalyzed by Binuclear Copper(II) Complexes in Aqueous Medium. ChemPlusChem 2015, 80 (1), 209-216.
41. Chen, W.; Tang, X.; Dou, W.; Wang, B.; Guo, L.; Ju, Z.; Liu, W., The Construction of Homochiral Lanthanide Quadruple-Stranded Helicates with Multiresponsive Sensing Properties toward Fluoride Anions. Chem. Eur. J. 2017, 23 (41), 9804-9811.
42. Baddi, S.; Palanisamy, A., Thermoreversible gelation of poly(urethane acyl-semicarbazides) carrying cycloaliphatic moieties and studies on selective adsorption of dyes from wastewater. Eur. Polym. J. 2018, 99, 90-101.
43. Sung, K.; Lee, A.-R., Synthesis of [(4,5-disubstituted-4H-1,2,4-triazol-3-yl)thio]alkanoic acids and their analogues as possible antiinflammatory agents. J. Heterocycl. Chem. 1992, 29 (5), 1101-1109.
44. de Faria, C. F.; Moreira, T.; Lopes, P.; Costa, H.; Krewall, J. R.; Barton, C. M.; Santos, S.; Goodwin, D.; Machado, D.; Viveiros, M.; Machuqueiro, M.; Martins, F., Designing new antitubercular isoniazid derivatives with improved reactivity and membrane trafficking abilities. Biomed. Pharmacother. 2021, 144, 112362.

What is claimed is:

1. A continuous process for preparing a compound of Formula (III) 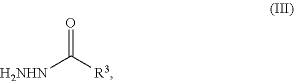
or a salt and/or solvate thereof, the process comprising:
(a) forming a first reaction stream comprising a compound of Formula (I) dissolved in an alcohol; 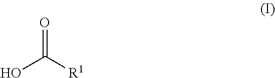
(b) flowing the reaction stream from step (a) through a continuous flow reactor at a temperature of about 100° C. to about 160° C. and at a flow rate of about 0.2 mL/min to about 5 mL/min;
(c) contacting the reaction stream from step (b) with a second reaction stream comprising a hydrazine source to form a combined reaction stream; and
(d) flowing the combined reaction stream from step (c) through a continuous flow reactor at a temperature of about 100° C. to about 160° C. and at a flow rate of about 0.2 mL/min to about 5 mL/min;
wherein
R¹ is Y or 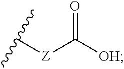
R³ is Y or 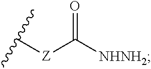
Y is an aryl, heteroaryl, alkyl, cycloalkyl, or heterocycloalkyl group;
Z is a divalent group selected from arylene, heteroarylene, alkylene, cycloalkylene, and heterocycloalkylene groups; and
R¹ and R³ are both Y, or R¹ and R³ are both not Y.

2. The continuous process of claim 1, wherein the alcohol in step (a) has a formula of R^{a}OH, wherein R^{a} is a C₁-C₆ alkyl.

3. The continuous process of claim 2, wherein the compound of Formula (I) is converted to a compound of Formula (II) in step (b), 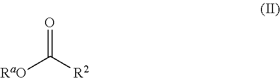
wherein:
R² is Y or 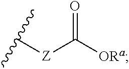
and
when R¹ and R³ are both Y, R² is Y; when R¹ and R³ are both not Y, R² is not Y.

4. The continuous process of claim 1, wherein the first reaction stream further comprises an acid, and wherein the acid is present in an amount of less than 0.5 equivalent relative to the amount of a compound of Formula (I).

5. The continuous process of claim 1, wherein the temperature in step (b) is about 135° C.

6. The continuous process of claim 1, wherein the flow rate in step (b) is about 2 mL/min or wherein the flow rate in step (b) is about 1 mL/min.

7. The continuous process of claim 1, further comprising cooling the reaction stream from step (b) to about 0° C. before step (c).

8. The continuous process of claim 1, wherein the second reaction stream in step (c) comprises an alcohol solvent.

9. The continuous process of claim 1, wherein the hydrazine source in step (c) is hydrazine hydrate.

10. The continuous process of claim 1, wherein the temperature in step (d) is about 125° C. or about 135° C.

11. The continuous process of claim 1 further comprising
(e) flowing the reaction stream from step (d) through a backpressure regulator; and
(f) cooling the reaction stream from step (e) to about 60° C.

12. The continuous process of claim 1, wherein the cooling step comprises flowing the reaction stream from step (e) through a continuous flow reactor at a temperature of about 60° C., and with a residence time of less than about 1 minute.

13. The continuous process of claim 1 further comprising collecting a reactor effluent exiting from the continuous flow reactor.

14. The continuous process of claim 1, wherein
the compound of Formula (I) is a compound having a formula of 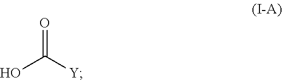
and
the compound of Formula (III) is a compound having a formula of

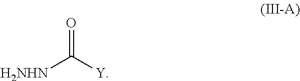

15. The continuous process of claim 1, wherein Y is isoxazolyl, phenyl, alkyl, or cycloalkyl.

16. The continuous process of claim 1, wherein Y is

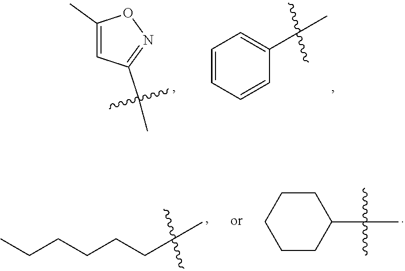

17. The continuous process of claim 1, wherein:
the compound of Formula (I) is a compound having a formula of 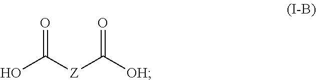
and
the compound of Formula (III) is a compound having a formula of

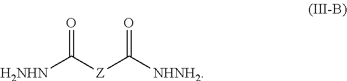

18. The continuous process of claim 1, wherein Z is phenylene, alkylene, or cycloalkylene.

19. The continuous process of claim 1, wherein Z is 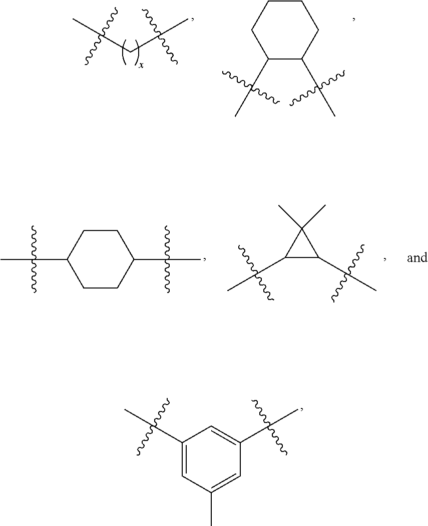
wherein x is an integer from 1-7.
